(19) United States
(12) Patent Application Publication
(10) Pub. No.: US 2010/0036295 A1
(43) Pub. Date: Feb. 11, 2010

(54) METHOD AND APPARATUS FOR FRACTIONAL DEFORMATION AND TREATMENT OF CUTANEOUS AND SUBCUTANEOUS TISSUE

(75) Inventors: Gregory B. Altshuler, Lincoln, MA (US); Robert R. Lopez, Boxford, MA (US); Andrei Erofeev, North Andover, MA (US); Christopher Gaal, Mansfield, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 12/538,712

(22) Filed: Aug. 10, 2009

Related U.S. Application Data
(60) Provisional application No. 61/188,308, filed on Aug. 8, 2008.
(60) Provisional application No. 61/211,829, filed on Apr. 3, 2009.

(51) Int. Cl. A61F 7/02; A61F 7/12

(52) U.S. Cl. 601/6; 601/15; 607/113; 607/105

(57) ABSTRACT

Devices and methods of treatment of tissue, such as skin tissue and subcutaneous tissue, with thermal control elements are disclosed. Some disclosed devices and methods employ local deformation of tissue in small areas. Devices and methods employing local deformation are used to produce fractional thermal treatments. Some devices and methods are external to a subject's skin others are beneath the subject's skin in the subcutaneous tissue.

### RELATED APPLICATIONS

This application claims the benefit of and priority to and is the non provisional application of U.S. Ser. No. 61/188,308 filed Aug. 8, 2008 entitled “Method and apparatus for fractional deformation and treatment of cutaneous and subcutaneous tissue” and U.S. Ser. No. 61/211,829 filed Apr. 3, 2009 entitled “Method and Apparatus for Treatment of Adipose Tissue by Cooling.”

### BACKGROUND OF THE INVENTION

1. Field of the Invention

The disclosure relates to the thermal and/or photothermal treatment of tissue. Thermal and/or photothermal treatment of tissue can be performed in conjunction with local deformation of tissue in small areas. Alternatively or in addition thermal and/or photothermal treatment of subcutaneous tissue can be performed via direct delivery into one or more fatty layer of tissue.

2. Background

Methods for reduction of fatty tissue, such as tumescent liposuction, are extremely popular. However, tumescent liposuction involves associated risks such as pain, bleeding, and/or anesthesia and drawbacks including associated downtime (time when the patient is unable to resume normal activity due to recovery from the procedure).

Each of heating and cooling of tissue at depth has individually proved useful for many treatments. The combination of heating and cooling applied intermittently to the skin surface (known as contrast therapy) is known and has been suggested for skin improvement, pain relief, inflammation reduction, and healing of injury.

### SUMMARY OF THE INVENTION

Of particular importance is the application of contrast therapy techniques for reducing fatty tissue such as subcutaneous fat deposits and treating cellulite (gynoid lipodystrophy). Non invasive and/or minimally invasive methods for reduction in fat tissue are desirable. In adults, the intracellular lipid content varies among cell types. Dermal and epidermal cells are relatively low in unsaturated fatty acids compared to the underlying adipocytes that form the subcutaneous fatty tissue. As a result, the different cell types, e.g., lipid-rich and other cells, have varying degrees of susceptibility to extreme temperatures e.g., hot and/or cold. In general, most cells can withstand colder temperatures than adipocytes.

In certain embodiments, controlled temperature changes may be used for non-invasive or non-destructive reduction of localized fat deposits. Adipocytes, e.g., in fat and/or adipose tissue, may be cooled or cyclically cooled and heated past their damage temperature, causing cell damage and/or destruction. Similar behavior can result from treatment of sebaceous glands; sebaceous glands can produce sebum in sebaceous or in hair follicles. The treated cells in sebaceous glands may undergo apoptosis, resulting in cell death. Treating sebaceous glands can potentially improved and/or avoid acne. The dead cells may then be removed or resorbed into the body, for example, by the body's phagocytic or lymphatic systems.

However, use of cooling or heating, either alone or in combination for treatment of conditions at depth, for example for skin improvement, cellulite improvement, fat reduction, and treatment of other conditions has been limited by the body's pain/discomfort tolerance and by the damage limits of treated organs and adjacent, especially cutaneous, tissue that need to be kept intact. The use of cooling or heating either alone or in combination for treatment of conditions at depth is also limited by the time that it takes for an effective temperature gradient to reach the desired target treatment area. Long treatment times limit adaptation by practitioners seeking the relatively brief treatment times conducive to treating multiple patients in a given day. A need therefore exists for improved methods and apparatus for thermal and photothermal treatment of tissue regions at depth, and in particular for treatment of skin and/or subcutaneous regions of tissue, which treatments provide improved treatment results, while reducing patient pain and discomfort. Protecting adjacent and other non-treated tissue from damage and/or controlling the damage imposed on adjacent and other non-treated tissue is also desirable. Thermal treatments include cooling or cycled cooling and heating. Thermal and/or photothermal treatment of tissue regions at depth must be capable of being conducted in treatment times that lead to practitioner adaptation.

In accordance with what is disclosed herein, in one aspect, a method for treating tissue, can include simultaneously compressing sub-volumes of the subject's skin and applying a thermal element proximal to the subject's skin to alter the temperature within a target depth of at least the depth of the reticular dermis. The target depth is substantially adjacent the sub-volumes and the sub-volumes are separated from one another by substantially uncompressed volumes of the subject's skin.

The reticular dermis may be located at about 0.25 mm below the surface of the skin, or at a deeper depth below the surface of the subject's skin. For example, the reticular dermis can have a depth that ranges from about 1 mm to about 3 mm in depth. The target depth can include at least one of the reticular dermis, subcutaneous fat and muscle. Subcutaneous fat can also be called or referred to as the hyperdermis. The thermal element can provide cooling, heating, a combination of cooling and heating, or a cycled combination of cooling and heating. In one embodiment, the thermal element includes a plurality of cooling elements adjacent to a plurality of heating elements. In accordance with the methods disclosed herein, the method can also include, applying treatment radiation to at least a portion of the sub-volumes. The one or more protrusions can both compress the sub-volumes and apply the thermal element proximal to the subject's skin.

In one embodiment, the tissue that is compressed and has its temperature altered can range from about 1% to about 50% of the treatment area, or from about 1% to about 30% of the treatment area.

Another aspect of the disclosure includes a device for selectively treating a subject's tissue can include a contact surface comprising two or more protrusions adapted to compress sub-volumes of a subject's skin such that the sub-volumes are separated from one another by substantially uncompressed volumes of the subject's skin. A thermal control element can provide temperature control to at least the two or more protrusions of the contact surface, the two or more protrusions are sized to alter the temperature of the subject's tissue at a target depth of at least the depth of the reticular dermis in the region of the compressed sub-volumes.

The substantially uncompressed volumes are not being directly compressed by the protrusions, rather, there may be some residual pressure in the areas between protrusions that causes some pressure and/or compression for example, due to the contact surface underlying the protrusions. The two or more protrusions are sized to have a depth that ranges from about 0.5 mm to about 25 mm and to have a width in contact with tissue that ranges from about 0.25 mm to about 50 mm, for example. The thermal control element can provide cooling, heating, a combination of cooling and heating, or a cycled combination of cooling and heating. The thermal control element can provide a plurality of protrusions adapted for heating adjacent to a plurality of protrusions adapted for cooling.

In some embodiments, a radiation source provide treatment radiation to at least one of the two or more protrusions and the radiation source is for applying treatment radiation to at least a portion of the sub-volumes. Suitable radiation sources can include lamp, light-emitting diode (LED), diode laser, solid state laser, gas laser, microwave or other electromagnetic radiation (EMR) sources. Other suitable energy sources that can provide treatment energy to at least one of the two or more protrusions include ultrasound energy.

In some embodiments, the contact surface is disposed on a wearable garment. Suitable wearable garments can be made from flexible materials, such as cotton, lycra, polyester etc. Other suitable wearable garments may be made from substantially inflexible materials such as, for example, a substantially inflexible polymer such as plastic, wood, paperboard, or metal. In some embodiments, the wearable garment is a patch, an adjustable cuff (e.g., a blood pressure cuff), pants, shirts, dresses, or undergarments. In one embodiment, wearing the wearable garment compresses the two or more protrusions against sub-volumes of the subject's skin.

In other embodiments of the device, the contact surface is disposed on a piece of furniture. In accordance with the device, positioning the subject on the piece of furniture compresses the two or more protrusions against sub-volumes of the subject's skin.

In some embodiments, the device is a vice having a first side, a second side, and a mechanism for compression disposed adjacent at least one of the first and the second side. The two or more protrusions are disposed on a surface of one or more of the first side and the second side, the one or more of the first side and the second side compress the two or more protrusions against sub-volumes of the subject's skin. The vice can further include a vacuum, which is applied to the device to increase the pressure applied to the subject's skin by the two or more protrusions. The mechanism for compression can be, for example, a plunger, a spring, a screw, a spacer. The mechanism for compression can be located adjacent to one or more of the first side and the second side (but not in between the first side and the second side); in such embodiments, the mechanism for compression pushes at least one of the first side and the second side into the opposing side. In another embodiment, the mechanism for compression is disposed between the first side and the second side and acts to pull the first and second side toward one another by, for example, a spring force.

In other embodiments, the device has a first roller, a second roller and a mechanism for compression disposed adjacent at least one of the first roller and the second roller. The two or more protrusions are disposed on a surface of one or more of the first roller and the second roller, the rollers are adapted to roll in opposing directions to thereby compress the two or more protrusions against sub-volumes of the subject's skin. The device can further include a vacuum, which is applied to the device to increase the pressure applied to the subject's skin by the two or more protrusions. The mechanism for compression can be, for example, a plunger, a spring, a screw, a spacer. The mechanism for compression can be located adjacent to one or more of the first roller and the second roller (but not in between the first side and the second side); in such embodiments, the mechanism for compression pushes at least one of the first roller and the second roller into the opposing roller. In another embodiment, the mechanism for compression is disposed between the first roller and the second roller and acts to pull the first and second roller toward one another by, for example, a spring force.

In some embodiments, at least one of the two protrusions has a solid shape. In other embodiments, at least one of the two protrusions has an at least partially hollow shape. In some embodiments, at least one of the two protrusions has shape selected from the group of square, rectangular, cylindrical, spherical, and grooved.

In another embodiment, the device includes a cup having two or more protrusions disposed on the inside surface of the cup. The cup is adapted to apply a vacuum to suction at least a portion of the subject's skin into the inside surface of the cup and to thereby compress the two or more protrusions against sub-volumes of the subject's skin.

Another aspect of the disclosure includes a method for selectively treating subcutaneous tissue, including inserting a cannula to a subcutaneous region of a subject's body and delivering cooling to the subcutaneous tissue such that the subcutaneous tissue being treated has a temperature within the range of from about −5 C to about 20 C. In one embodiment, the cannula delivers cooling to the subcutaneous tissue by thermal conduction. In another embodiment, the cannula delivers a cooling agent to the subcutaneous tissue. The method can also include venting at least a portion of the cooling agent from the subcutaneous region. In some embodiments, the cannula circulates a cooling agent to the subcutaneous tissue and external to the body of the subject.

### DESCRIPTION OF THE FIGURES

FIG. 1 shows four curves depicting depth in mm on the x-axis and temperature in ° C. on the y-axis over a variety of time periods of application of a cooling panel to the skin surface ranging from curve (1) after one minute, curve (2) after five minutes, curve (3) after ten minutes, and curve (4) after thirty minutes.

FIG. 2 shows the calculated temperature dynamics of the dermis-subcutis junction at 2.5 mm depth and in subcutaneous fat at 7.5 mm depth resulting from a constant surface temperature of 0° C., the x-axis shows time in seconds and the y-axis shows temperature in ° C.

FIG. 3 has two x-axis, one of which shows the display temperature in ° C. and the other of which shows the surface temperature in ° C., the y-axis shows time in seconds, and features two curves one shows the time for the onset of unpleasant sensation and the other shows the time for the onset of pain.

FIG. 4 shows a contact tip having multiple sub-regions (e.g., protrusions) having a square shape.

FIG. 5 shows a contact tip having multiple sub-regions (e.g., protrusions) having a rectangular shape.

FIG. 6 shows a contact tip having multiple sub-regions (e.g., protrusions) having a grooved shape.

FIG. 7A shows a fractional contact tip having a protrusion being pressed into a subject's skin.

FIG. 7B shows a non fractional contact tip being pressed into a subject's skin.

FIG. 8A shows a plot of the change in tissue temperature over time when the tissue is exposed to an ordinary contact tip (dotted line plot) having a temperature of 5° C. and when the tissue is exposed to a fractional contact tip (solid line plot) having a temperature of 5° C.

FIG. 8B shows a plot of the change in tissue temperature over time when the tissue is exposed to a fractional contact tip having a temperature of 5° C. in the presence of tissue having: (1) a fixed water content (dotted line plot) and (2) a variable water content (solid line plot).

FIG. 9A shows a diagram of a chair having one or more fractional contact tips.

FIG. 9B shows a fractional contact tip that includes a combination of protrusions for cooling and for heating.

FIG. 9C shows another close up view of protrusions.

FIG. 10A shows the seat of a chair having one or more fractional contact tips.

FIG. 10B shows one or more fractional contact tips having sold protrusions.

FIG. 10C shows one or more fractional contact tips having at least partially hollow protrusions.

FIG. 11 shows a cup with one or more protrusions on its inner surface, the protrusions for providing thermal control, the cup capable of employing vacuum against the subject's skin surface.

FIG. 12 shows a device that works like a vice having a first side and a second side, each side having protrusions on the exterior surface, a spring is disposed between the first side and the second side and the subject's skin is compressed between the first side and the second side including by the protrusions.

FIG. 13 shows a device that works like a vice having a first side and a second side, each side having protrusions on the exterior surface, a spring is disposed between the first side and the second side and the subject's skin is compressed between the first side and the second side including by the protrusions the device employs vacuum to provide contact between the subject's skin, the first side, the second side and the protrusions.

FIG. 14 shows a device that includes at least two opposing rollers, each roller having one or more protrusions disposed on the external surface, a spring is disposed between each of the opposing rollers, and the subject's skin is compressed between the rollers and including by the protrusions.

FIG. 15 shows a device that includes at least two opposing rollers, each roller having one or more protrusions disposed on the external surface, a spring is disposed between each of the opposing rollers, and the subject's skin is compressed between the rollers and including by the protrusions, the opposing roller device employs vacuum to provide contact between the subject's skin, the opposing rollers and the protrusions.

FIG. 16A shows a cross section of human tissue including a layer of skin, a layer of fat below the layer of skin, a layer of muscle below the fat layer and, optionally in some embodiments another layer of fat below the layer of muscle.

FIG. 16B shows a second of human tissue with a cannula that delivers a cooling agent to human tissue, another cannula that delivers a cooling agent to human tissue and another cannula employed to aspirate liquid and/or gas from the subject's body, and a multi lumen cannula that includes a delivery portion that delivers a cooling agent to human tissue and an aspiration portion to aspirate liquid and/or gas from the subject's body.

FIG. 17 shows a cannula for delivering a cooling agent to a subject's body.

FIG. 18 shows two cannulas for delivering cooling agent to a subject's body and for venting/aspirating liquid and/or gas from a subject's body.

FIG. 19 shows closed loop circulation of cooling agent in a cannula.

### DETAILED DESCRIPTION OF THE INVENTION

Treatment of tissue (e.g., fat tissue) with cooling can be employed for “lipolysis,” or the process of breaking down fat in the body to contribute to a reduction of fat cell(s) and/or fat tissue in a region of tissue being treated.

Cooling of the fat tissue can include cooling to a temperature below normal body temperature, and preferably below the phase transition temperature of at least some fraction of the lipid content of fatty cells. The phase transition temperature of at least some of the fraction of the lipid content of fatty cells is substantially higher then the freezing temperature of water-containing tissue. Optionally, cooling of the fat tissue can be preceded by or followed by heating the fat to a temperature below its damage threshold

Adipose tissue comprising adipocytes can be selectively disrupted while avoiding substantial and/or all collateral injury to the surrounding non lipid-rich tissue (e.g., dermal and epidermal tissue) by controlling the temperature and/or pressure applied to the respective tissues. In addition, by controlling the temperature and/or pressure applied to the respective tissues, disruption of adipose tissue comprising adipocytes can be can be accomplished together with controlled injury to the surrounding non lipid-rich tissue (e.g., dermal and epidermal tissue).

### Cooling

Cooling can be employed to selectively disrupt adipocytes, sebocyte and sebaceous glands by creating a low temperature gradient within a local region sufficient to selectively disrupt and thereby reduce the adipocytes contained within the local region. The cooling temperature gradient can be provided to the local region by exposing the subject's skin to a cooling element. The cooling energy can travel to the target lipid-rich tissue via the subject's tissue. Optionally, a cooling element temperature can provide a temperature gradient selected to not treat or have non substantial impact on non lipid-rich cells within the temperature gradient and/or in the subject's skin. For example, any or any substantial treatment, damage, or disruption to non adipocytes may be avoided. Non-substantial treatment, damage, or disruption can include, for example, inflammation, irritation or swelling. The lipid-rich adipocytes may be contained in, for example, subcutaneous adipose tissue (adipocyte), sebaceous glands and sebocytes. Cooling the local region at a temperature sufficient to selectively disrupt adipocytes enables local reduction of adipose tissue. Cooling the local region around sebaceous glands at a temperature sufficient to selectively disrupt sebocyte and/or sebaceous gland(s) enables acne reduction. The temperature gradient can selectively disrupt and thereby reduce the adipocytes within the local region. In some embodiments, the cooling element temperature can provide controlled damage to other organ(s) (e.g., epidermis and/or dermis).

Selective disruption of adipocytes appears to result from localized crystallization of highly saturated fatty acids upon cooling at temperatures that do not induce crystallization of water in other cells. Accordingly, crystallization damage of non lipid-rich cells or molecules, such as fibroblasts, blood cells, collagen-fibers, can be avoided at temperatures that induce crystal formation in adipocytes. Crystallization of all or a portion of an adipocytes disturbs, damages and/or destroys the adipocyte. As a result of crystallization the function of the fatty cells is at least somewhat impaired and/or destroyed. After crystallization damage the fatty cells are more readily removed from the fatty tissue area. It is also believed that cooling can induce lipolysis (e.g., metabolism) of adipocytes, further enhancing the reduction in subcutaneous adipose tissue. Lipolysis may be enhanced by local cold exposure inducing stimulation of the nervous system. Crystallization of lipid in adipocytes can occur at a temperature below about 37° C., for example from about 20° C. to about 0° C., or from about 10° C. to about 20° C.

Generally, the time for changing the temperature at the skin surface (e.g., cooling) must be long enough to allow the temperature gradient to flow from the epidermis to the dermis to the subcutaneous adipose layers in order to achieve the desired temperature at the adipose layer that provides the treatment. The subcutaneous adipose layer can include adipose tissue, which can be made up of lipid rich tissue and connective tissue.

When the subcutaneous adipose is cooled to a temperature below the temperature for lipid crystallization (e.g., below 37° C., for example from about 20° C. to about 0° C.), the latent heat of freezing for these lipids must also be removed, by diffusion. In some embodiments, the skin surface cooling temperature and cooling time can be adjusted to control depth of treatment, for example the anatomical depth to which subcutaneous adipose tissue is affected. Heat diffusion is a passive process, and the body core temperature is nearly always close to 37° C. Therefore, generally, for at least part of the time during which cooling is performed the skin surface temperature must be lower than the desired target temperature for treatment of the adipocytes in the target region.

### Cycled Cooling and Heating

In another embodiment, the adipocytes in adipose tissue are exposed to one or more cycle of cooling and heating. For example, the adipocytes in fatty tissue may be cooled to a temperature below normal body temperature, and preferably below the phase transition temperature of at least some fraction of the lipid content of the lipid-rich fatty cells (e.g., less than about 37° C. or from about 0° C. to about 20° C.). The temperature of lipid crystallization is higher then the freezing temperature of water, which is about 0° C.-10° C. The cooling step may be preceded or followed by heating the fat to a temperature below its damage threshold (e.g., from about 20° C. to about 50° C., or about 40° C.). Triglycerides (which constitute the largest fraction of lipids in human fatty tissue) undergo a series of phase transitions when their temperature changes from normal body temperature to either a lower or a higher temperature. Specifically, several crystalline forms of triglycerides can exist. These crystalline forms are (in the order of increasing stability): α, β′, and β. The β crystals are also significantly larger in size (as large as a needle having a dozen microns in length) than the α and β′ crystals. Crystal formation can be the reason for adipocyte dysfunction, shrinkage, or destruction resulting from mechanical stress on cell structure and/or destruction of cell metabolism. Formation of β crystals can be the primary mechanism to treat adipocytes. When triglycerides are cooled from normal body temperature, formation of α-crystals takes place. In order to produce more stable forms, β′ first and β second, the crystallized triglycerides can be heated. Further heating leads to complete melting of all crystalline forms.

In one embodiment, to initiate formation of β-crystals in adipose cells, first, the fatty tissue is cooled to a lower-than-normal temperature (e.g., a temperature to form α crystals), which is in the range between about 0° C. and about 37° C. This cooling step results in formation of α-crystals. Then, the tissue is heated back to a temperature T_{β}>T_{α} but below 37° C., causing formation of β′- and β-crystals. Optionally, the tissue is heated to an even higher temperature (including above 37° C.) in order to destroy envelops containing lipid droplets in the adipocyte and to melt the crystals. This cooling and heating process can be repeated for a selected number of cycles. The cycle of formation of α-crystals, formation of β′- and β-crystals, and the melting of the crystals exerts mechanical stress on the adipocyte structure. The expected final result is dystrophy and decrease in volume of fatty tissue. This process takes place for all temperatures in the range of from about 0° C. to about 37° C., but for a lower T_{α} this process is more effective. Thermal activation of lymph systems in subcutaneous fat can also be used to treat cellulite by removing proteins from extra cell spaces.

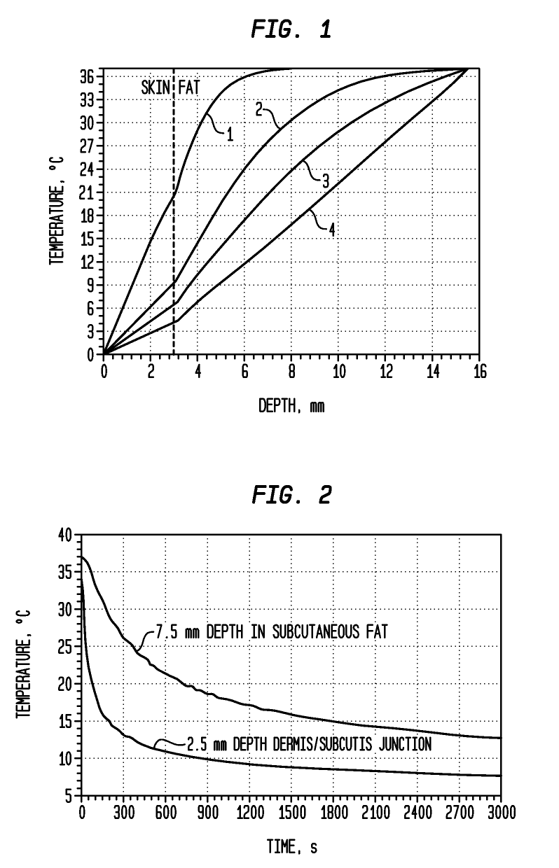

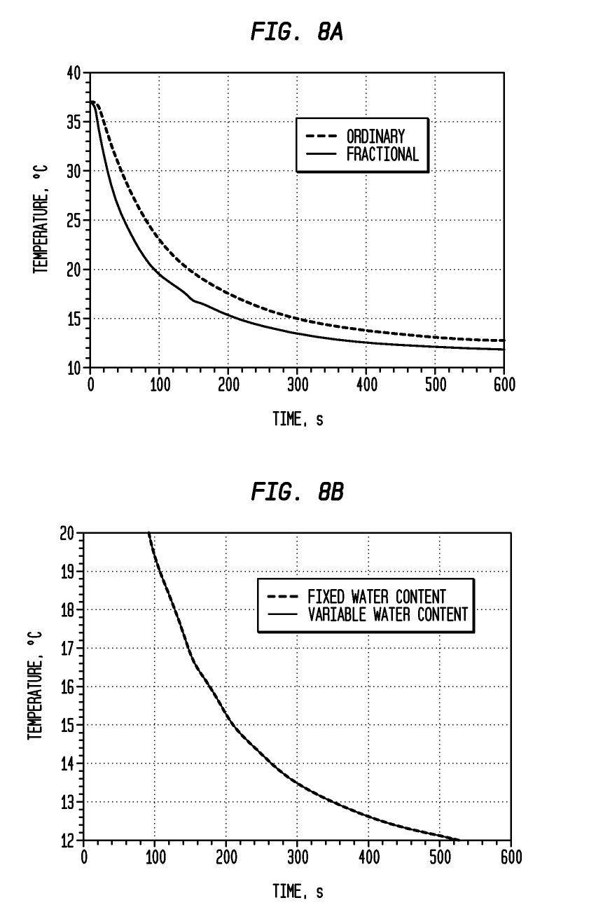

Application of a cooling agent or a cooling device (e.g., the non fractional contact tip 800 shown in FIG. 7B) to the skin surface causes the temperature of skin and the subcutaneous region or subcutis to drop gradually, as illustrated by FIG. 1. In FIG. 1, curve (1) is after one minute, curve (2) after five minutes, curve (3) after ten minutes, and curve (4) after thirty minutes of application of a cooling panel to the skin surface. The depth of the skin/fat or dermis/subcutaneous boundary, shown at 3 mm in FIG. 1, will vary depending on a number of factors including the patient and the portion of the patient's body being treated. The rate of cooling and the final temperature depend on the depth of the target and the temperature of the skin surface.

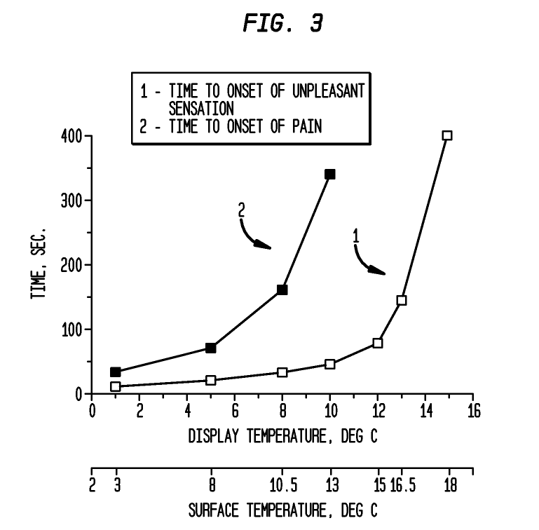

FIG. 2 shows the calculated temperature dynamics of the dermis-subcutis junction at 2.5 mm depth and in subcutaneous fat at 7.5 mm depth resulting from a constant surface temperature of 0° C. Substantial cooling of targets in the skin can be achieved in the time range between 10 seconds and 300 seconds. Deeper targets in subcutaneous fat needs cooling times in the range of between about 2 min and about 30 min.

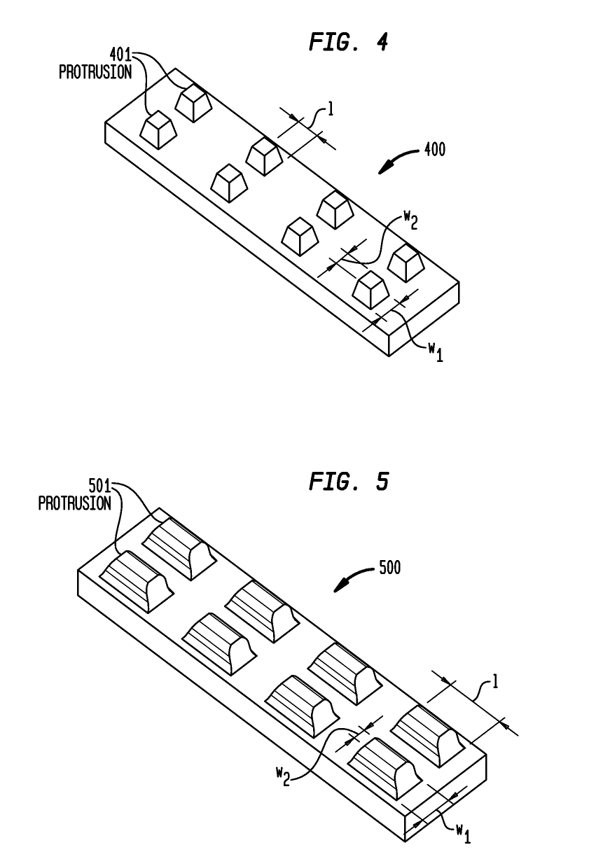

In practical use, cold exposure time is limited by the onset of unpleasant and, subsequently, painful sensations by the subject. FIG. 3 illustrates the dependence of these onset times on the temperature at the skin surface. As a result, the practical application time may be insufficient to achieve a desired therapeutic effect. Cooling time can be shortened by simultaneously coupling into the skin pressure or acoustic waves or by intensive massage of cooled skin. The acoustic waves or mechanical massage can increase the heat conductivity of the skin and subcutaneous fat by forced convection of inter-cellular water.

Depending on the surface temperature and the duration of application, a number of processes can be initiated in the fatty tissues and the other tissues, the processes can include, but are not limited to: phase transitions in lipids and changes in regulatory functions of the adipocyte(s). In particular, lower temperatures may suppress activity of Alpha2 receptors, which inhibit adenylate cyclase and cyclic AMP through Gi protein and thus decrease lypolisis rate. This can lead to long-term atrophy of fatty cells after cold exposure. This can also lead to an increase of ion concentration in intracellular water. Such an increase in intracellular water is caused by partial binding of free water in the course of fat crystallization. Transition of water into a bound state has been demonstrated spectroscopically. As a result, the concentration of ions in the remaining free water increases. Once the ion concentration exceeds a critical level, irreversible damage to the vital mechanisms of the cell can occur, types of irreversible damage include (1) water crystallization in tissues (2) induction of apoptosis (3) tissue necrosis (4) stimulation of thermogenesis (5) remodeling of vascular and lymph vessels and (6) temporal or permanent dysfunction of follicles, sebaceous and sweat glands. Thermal cycling, which includes both cooling and heating phases on the external surface of the skin, has been used to lessen the pain and/or discomfort and unwanted tissue damage outside the target treatment region. Where thermal cycling, comprised of cooling and heating phases, is employed pain/discomfort and/or unwanted tissue damage outside the target region may be reduced or eliminated. It should be emphasized that the thermal inertia of tissue prevents rapid propagation of a heat front from the skin surface to a desired treatment depth (or vice versa). Thus, such prior treatments are limited by, for example, the risk of irreversible tissue damage and/or the extended length of time required to reach the depth of desired tissue treatment.

External treatment approaches can include deep-penetrating electromagnetic or acoustic radiation, which may be used to distribute heat sources within tissue. This allows increasing the treatment time substantially and enables acceptable therapeutic effect while retaining a non-invasive procedure that complies with the patient's comfort requirements.

Beneficial effects of thermal cycling are not limited to treatment of fatty tissue. Thermal cycling initiates a number of biophysical and biochemical responses at molecular, cellular, tissue, and organ levels, including (but not limited to): modulation of cell membrane's permeability and, therefore, inter-membrane transport and exchange between intra- and interstitial compartments; induction of thermo-mechanical stress in the target (for example, malignant) cells, leading to cell death through either necrosis or apoptosis; changes in elasticity and permeability of vessel walls; changes in blood rheology; stimulation of tissue regeneration, including new collagen generation in skin and subcutis; and changes of toxin structure in the interstitial fluid making them amenable for easy removal by lymphatic systems.

As a result, thermal cycling can be used for treatment of a wide range of conditions, involving skin, subcutaneous fat, connective tissues, blood and lymph vasculature, muscles, bones, and other internal organs. Thermal cycling can be employed by approaches that range from external to the subject's body (e.g., via the external surface of the skin) to internal to the subject's body.

### Thermal Element

In order to practice the treatment of fatty tissue, a thermal element is employed to treat the target region. The thermal element can be solely for cooling or can cycle cooling and heating. The thermal element can be employed as a thermal control element. The thermal element can be a cooling element such as a contact tip or a contact agent that is applied in contact with or proximal to the subject's skin in a target region where subcutaneous adipose tissue reduction or acne reduction is desired. The thermal element can be a cooling agent that is applied externally, internally (e.g., by injection), or a combination thereof. Contact with the cooling element can create a temperature gradient within the target region sufficient to selectively disrupt adipocytes and/or sebaceous glands therein. Application of the cooling element to the subject's skin may be repeated a plurality of times until the desired reduction in subcutaneous adipose tissue has been achieved. Where the cooling element is a contact tip it may be coupled to or contain a cooling agent. Cooling elements of the present invention can contain cooling agents in the form of a solid, liquid or gas. Solid cooling agents can include, for example, thermal conductive materials, such as metals and/or metal plates and solid cooling agents can also includes glasses, gels and ice or ice slurries. Liquid cooling agents can comprise, for example, saline, glycerol, alcohol, or water/alcohol mixtures, for example. Where the cooling element includes a circulating cooling agent, preferably the temperature of the cooling agent is constant. Salts can be combined with liquid mixtures to obtain desired temperatures. Gasses can include, for example, cold air or liquid nitrogen. In one embodiment, the cooling element is applied such that direct contact is made with a subject, via either the agent or the element. In another embodiment, direct contact is made via the agent alone. In yet another embodiment, no direct contact is made via either the agent or the element and cooling is a carried out by proximal positioning of the cooling element and/or agent. Preferably, the temperature of the cooling agent is less than about 20° C.

The cooling agent can be applied in a pulsed or continuous manner. The cooling element and/or agent can be applied by all conventional methods known in the art, including topical application by spray of a cooling agent in liquid form, gas or particulate solid material. Cooling may be applied externally or, alternatively, cooling elements and/or agents can also be applied subcutaneously by injection or other conventional means. For example, the cooling agent can be applied directly to the subcutaneous tissue and then either removed after contact or left in the subcutaneous tissue to achieve thermal equilibration and therefore cooling of the lipid-rich tissue (e.g., subcutaneous injection of a liquid cooling agent or of small cooling particles, such as pellets or microbeads).

Where the thermal element cycles cooling and heating, the cooling unit can be a thermoelectric element, an enclosure with cooling agent, a stream of cold gas (or liquid) or other cooling unit known in the art. Phase-changing materials can also be used for cooling. Skin surface temperature during the cooling phase should be maintained within the range of from about −5° C. to about 30° C. or from about 0° C. to about 20° C. Tissue temperature in the heating phase should be maintained in the range of from about 25° C. to about 55° C., or from about 25° C. to about 40° C., or from about 35° C. to about 45° C. In one embodiment of the invention, optical radiation is used in the heating phase of the cycle. In another embodiment, electromagnetic radiation (EMR) is used on the heating phase of the cycle. In this embodiment, the energy source can be a laser, an LED, a lamp (discharge, halogen or other), or a combination or an array thereof. For example, the optical radiation can travel through or around the thermoelectric element. The spectral composition of the source can be either narrow- or broad-band, with the range of wavelengths between 400 nm and 2000 nm. Spectral filtration can be used for further modifying spectral composition of the beam in order to achieve optimal penetration. The wavelengths used for a particular application will depend on the target tissue, the depth of the tissue and other factors. In one embodiment, the light source is operated in the continuous wave (CW) mode, with a preferred irradiance at the skin surface in the range between 0.1 and 100 W/cm². The thermal cycle is organized in such a way as to maximize efficacy of treatment. Typically, duration of the cooling phase can be between about 10 seconds and about 30 minutes, whereas duration of the heating phase can be between about 1 second and about 4 minutes.

### Fractional Treatment

In the cosmetic (e.g., skin rejuvenation) field, for the treatment of various skin conditions, methods and devices have been developed that irradiate or cause damage in a portion of the tissue area and/or tissue volume being treated. These methods and devices are known as fractional technology. Fractional technology is thought to be a safer method of treatment of skin for cosmetic purposes, because tissue damage occurs within smaller sub-volumes within the larger volume of tissue being treated. The tissue surrounding the sub-volumes is spared from the damage. Because the resulting sub-volumes are surrounded by neighboring healthy tissue the healing process is thorough and fast. Furthermore, it is believed that the surrounding healthy tissue aids in healing the tissue damaged by the treatment. Examples of fractional treatment devices that have been used to treat the skin using non-ablative cosmetic procedures such as skin resurfacing include the Palomar® 1540 Fractional Handpiece, the Solta Medical Fraxel® SR Laser and similar devices by ActiveFX, Alma Lasers, Iridex, and Solta Medical.

Without being bound to any single theory it is believed that use of a fractional treatment strategy for cooling and/or for cycling cooling and heating sub-volumes of tissue within a larger target volume of tissue being treated can likewise aid in healing the tissue damaged by the treatment. Fractional treatment via cooling and/or via cycled cooling and heating can also be employed to produce controlled damage to the epidermis, dermis, and/or hypodermal layers of the subject's tissue. The controlled damage can promote collagen formation when the sub-volumes of treated tissue heal. Alternatively or in addition, fractional treatment via cooling and/or via cycled cooling and heating may also be employed at a depth to treat subcutaneous fatty tissue and/or muscle tissue. Treatment at a depth (e.g., treatment of subcutaneous fatty tissue and/or muscle tissue) and treatments closer to the surface (e.g., treatment of epidermis, dermis, hypodermis) can occur simultaneously, e.g., during a single cooling treatment, or separately.

Fractional treatment of a target region of tissue is believed to limit, reduce, and or eliminate the pain/discomfort sensed by the body being treated relative to non-fractional treatment of the same volume of tissue being fractionally treated. The body can better tolerate any pain/discomfort sensed when the treatment is conducted fractionally as compared to the same treatment conducted in a non fractional manner. In addition, with fractional treatment, the neighboring healthy tissue enables the treated tissue to recover more readily via the multiple non-treated healthy tissue “sides” that surround the treated sub-volume of tissue.

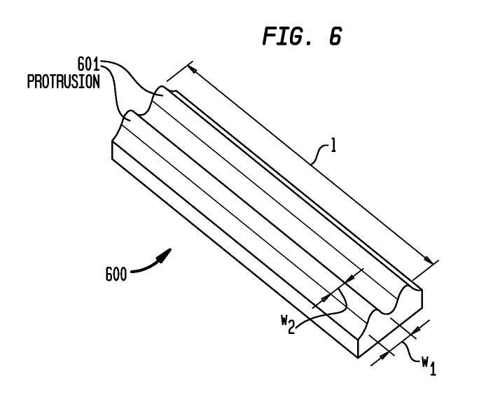

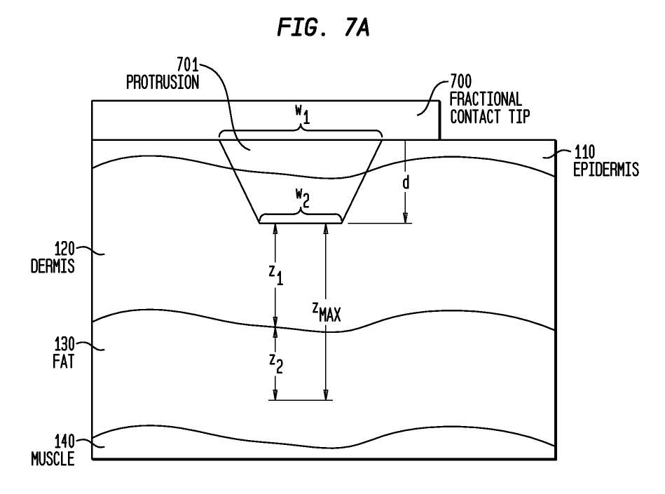

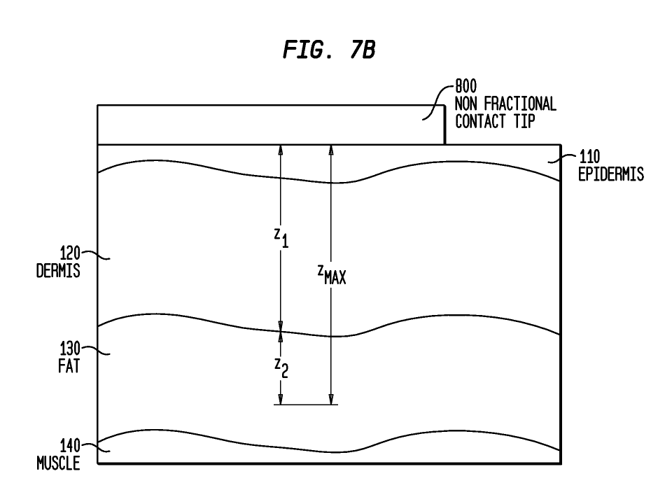

The cooling and/or heating can be applied to the patient's skin fractionally via a contact tip. The contact tip can have any of a number of fractional configurations. Each of the multiple sub-regions can have the same shape or a single contact tip can have a variety of sub-region shapes. The multiple sub-regions for fractional treatment can be positioned in any of a number of patterns (e.g., circular or rectangular). Suitable sub-region protrusion shapes include, for example, squares 401 (FIG. 4), rectangles 501 (FIG. 5), and grooves 601 (FIG. 6). The sub-region shapes and patterns may be selected to suit the region of the body to be treated and/or the quantity of fatty tissue to be treated, for example. One or more of the exemplary contact tips 400, 500, or 600 shown in FIGS. 4-6 can be adapted to provide multiple sub-regions for cooling alone, multiple sub-regions combining cooling and heating with the some sub-regions for cooling and other sub-regions for heating, and/or multiple sub-regions for cycling cooling with optical energy heating. The one or more sub-regions can be in the form of protrusions that extend to a depth.

FIG. 7A shows a cross section of sub-region of a protrusion of a contact tip similar to the contact tips shown in FIGS. 4, 5 and 6 or tips with other shape for example cylindrical tips being pressed into a subject's skin such that it presses the subject's epidermis 110 and into the subject's dermis 120 to the depth (d). In one embodiment, where cooling and heating is cycled, one or more of the sub-regions (e.g., cross section of sub-region shown in FIG. 7A) may have one or more integrated optical waveguides or windows that enable dual use of a sub-region for fractional cooling and for fractional light based treatment. It is possible that a single sub-region enables one or more light based transmission there through.

Referring still to FIG. 7A, each of the sub-regions of the fractional contact tip 700 has a depth (d) in addition to its length (l) which is a cross section perpendicular to the length (l) and a first width (w₁) and a second width (w₂) shown, e.g., in FIGS. 4-6. The first width (w₁) can vary in the range from about 0.25 mm to about 50 mm. The first width w₁ is related to the depth (zₘₐₓ) of cooling or heating, which can be in the range of from about 0.5 mm to about 25 mm and w₁ is from about 0.5*(zₘₐₓ) to about 3*(zₘₐₓ). The second width (w₂) is smaller than w₁ and can to be in the range of from about 0.5*(zₘₐₓ) to about 2*(zₘₐₓ).

For example, the fractional contact tip 600 and 700 shown in FIGS. 6 and 7 has at least one sub-region having a depth (d). The depth of treatment created by one or more of the sub-regions of the fractional contact tip (having depth d) ranges from about 200 microns (μ) to about 25 millimeters (mm), or from about 0.5 mm to about 2 mm. The depth of the fractional contact tip may be selected to compress the tissue area being treated. For example, referring to FIG. 7A, the depth and/or exerted pressure may be selected to enable compression of epidermis 110 tissue, dermis 120 tissue and into the subcutaneous fat 130 tissue. In another embodiment, the contact tip fractional depth and/or exerted pressure is selected to enable compression of only the epidermis 110 tissue and the dermis 120 tissue. The sub-regions of the fractional contact tip 700 may be referred to as one or more protrusions 701 that have a depth (d) that measures from about 200μ to about 10 mm, or from about 0.5 mm to about 2 mm in depth. In one embodiment, the fractional compression is referred to as micro compression.

When a small area of tissue is deformed by compressing or applying pressure to the area or sub-volume of tissue when a thermal and/or photothermal treatment is applied, the penetration of light, heat or cool energy into the tissue is greater than the penetration of the same energy into tissue that is not so deformed. This phenomenon can be used, in particular, to improve fractional thermal and/or photo thermal treatment of tissue and to develop new such treatments. However, the principle is also applicable to non-fractional treatments, where the deformation of a number of small areas of tissue can be used to improve the penetration of energy in non-fractional applications that treat a relatively larger area relative to the size of the deformed areas.

Use of a pressure or compression strategy for cooling, combining cooling and heating, and/or for cycling cooling and heating of areas or sub-volumes of tissue being treated at a depth can limit, reduce, and or eliminate the pain/discomfort sensed by the body being treated relative to non compression treatment. Because the body senses the applied pressure in addition to the cooling and/or the cycled cooling and heating, application of pressure and/or compression can limit, reduce and/or eliminate the sensation of pain or discomfort sensed by the body being treated relative to a treatment conducted in the absence of fractional pressure and/or compression. Fractional compression reduces or blocks the pain sensation and allows delivery of more cold, heat or light energy without pain. Where pressure and/or compression are applied in a fractional manner, neighboring healthy tissue enables the treated tissue to recover more readily via the multiple non-treated, healthy tissue regions or “sides” that surround the treated tissue.

The depth of the fractional contact tip may be selected to enables treatment of a target depth of at least the depth of the reticular dermis. The reticular dermis can be at about 0.25 mm below the surface of the subject's skin, or it can be deeper. For example, the reticular dermis can be at a depth below the subject's skin that ranges from about 1 mm to about 3 mm, for example.

For the practical application of any treatment it is critical that the treatment time is optimized to reduce the time required for treatment, because a reduced treatment time enables the practitioner to treat each subject quickly and be able to maximize the number of subjects treated in a single day. The cooling time is limited by the time it takes for the drop in temperature gradient to flow through the tissue to the target region of dermis or fatty tissue. The delta in cooling time is a function of the properties of the treatment device (e.g., the tip material such as a sapphire cooling agent), the temperature of the surface of the treatment device contacting with tissue, and the thermal properties of the tissue (e.g., epidermis, dermis, fat, and/or muscle) being treated, thermal diffusion, density, and specific heat capacity and the distance between the skin surface and the treatment area. Micro compression of the skin displaces water from dermal tissue and shortens the distance between the skin surface and the treatment area. The distance between the skin surface and the treatment area can be shortened by up to two times resulting is a faster extraction of heat from the compressed area and thereby decreasing the treatment time. The temperature and time of exposure of the treatment device to the tissue impacts the delta in cooling time. The treatment device can be a contact cooling device made from any of a number materials including sapphire, copper, or aluminum. In an embodiment where cycling of cooling and heating is desired, in a fractional contact tip, one or more sub-regions can include aluminum with integrated optical waveguide or window, for example, where it is desirable combine fractional cooling with fractional light based treatment.

Constraints on cooling treatment include that there is a limit to the amount of cold that the epidermis will tolerate before the epidermis is damaged and/or before the subject experiences discomfort from the treatment. However, where the cooling is introduced to the epidermis according to a fractional technique, non-treated or undamaged tissue surrounds any sub-volume of epidermis and/or dermis tissue damaged by the treatment by exposure to cold during the cooling treatment. The undamaged tissue aids in the healing of any fractionally damaged sub-volume of tissue. The fractional treatment methodology lessens the likelihood of causing permanent damage to the tissue. As a result, during fractional treatment, the sub-regions of skin (e.g., the epidermis and the dermis) can be exposed to a lower temperature and/or to a lower temperature (e.g., about −10° C. to about 0° C.) for a longer period of time without risk of permanent damage to the skin as compared to the same treatment conducted in a non fractional manner. When a fractional cooling treatment is employed the sub-region of tissue may be treated more severely, because it is on such a small scale (e.g., a micro scale) and is surrounded by healthy tissue to help the damaged tissue to recover. During a fractional cooling treatment the subject can tolerate skin exposure to a lower treatment temperature and/or a lower treatment temperature for a longer period of time as compared to the same treatment conducted in a non fractional manner. As a result the cool temperature can reach adipocytes more rapidly fractionally as compared to a treatment conducted in a non fractional manner. Use of fractional cooling is thereby expected to reduce the time required to treat the target lipid-rich fatty tissue.

The treatment (cooling application) time t_{cool} should be long enough to remove heat from the layer 0<z<zₘₐₓ, where zₘₐₓ is maximum treatment depth. This time t_{cool} is given by the Formula 1:

$${t}_{cool} = \frac{A \cdot {z}_{max}^{3}}{{α}_{1} \cdot {z}_{1} + {α}_{2} \cdot {z}_{2}} \qquad ( Formula 1 )$$

where z₁ is the depth into dermis and z₂ is depth into subcutaneous fat, and z₁+z₂=zₘₐₓ is depth of treatment.

A is a dimensionless coefficient that is derived from an analytical heat diffusion equation based in part on the geometric form factor of the treated skin. A ranges from about 0.25 to about 10.

α₁ and α₂ are thermal diffusion coefficient of the dermis and fat, respectively. One can see from this formula that by decreasing the depth into the dermis, z₁ the time of cooling can be decreased.

It is desirable to optimize t_{cool} to provide a reduced cooling time, because reducing the cooling time t_{cool} helps to optimize the throughput of the treatment, namely how quickly the treatment can take place.

Referring now to FIGS. 7A and 7B and the formula for t_{cool} the optimal cooling time is governed in part by the relationship between different depths of the tissue (e.g., skin) the dermal layer z₁ and the target depth z₂, namely the depth of subcutaneous fat 130 target treatment. The optimal cooling time may be evaluated using the depths of the dermal layer z₁ and the depth of the specified target z₂.

When cooling begins the temperature at z₁ falls abruptly, whereas the temperature at z₂ remains nearly fixed. When the thermal change reaches the target depth z₂ then the temperature of the target depth z₂ begins to decrease. The temperature at the dermal layer z₁ reaches a steady state and stops changing. The calculation for t_{cool} to determine the cooling time depends on the distance between the dermal layer depth z₁ and the target depth z₂. In fact, because the relationship between each of the dermal layer depth z₁ and the target subcutaneous fat layer depth z₂ sum to provide zₘₐₓ, which is to the power of two, there is a great potential to reduce the optimum time for cooling when you fractionally compress the tissue, via for example the protrusion of a fractional contact tip, thereby moving the dermal layer depth z₁ closer to the target subcutaneous fat layer depth z₂ (via, e.g., fractional micro compression)

In contrast, referring to FIG. 7B, when treating the same target region of tissue as shown in FIG. 7A, but with a non fractional contact tip the delta between the dermal layer depth z₁ (non fractional) and the target depth z₂ is larger, which will result, in accordance with the t_{cool} calculation, in a longer amount of time to cool the target region than if the cooling was applied fractionally. Thus, cooling coupled with fractional compression can reduce the time required for cooling the target region.

In addition, referring still to the formula for T_{cool}, α is the diffusion coefficient of human tissue and is related to the water concentration contained in the tissue. Compression of tissue reduces the water concentration in the compressed region and may also play a role in the time required to cool the tissue. Determination of the overall change in the diffusion coefficient α due to compression and cooling is not straight forward since the diffusivity of human tissue is a function of conductivity (κ), density (ρ), and heat capacity (c) and this relationship is represented by Formula 2.

α=κ/(ρ*c) (Formula 2)

Generally, human skin tissue is believed to contain from about 65% to about 75% water. As tissue is compressed, at least a portion of the water in the tissue is believed to be moved out of the region of compressed tissue. As a result of the drop in water content it is expected that the tissue conductivity (κ), density (ρ) and tissue heat capacity (c) will be impacted.

Amongst the other considerations described herein, the desired pressure exerted on the tissue via the fractional contact tip may be determined based on the impact of compression on the dermal tissue thermal diffusion coefficient α₁ and the subcutaneous fat tissue thermal diffusion coefficient α₂.

Applying compression to tissue at the external surface of a subject's skin with a fractional contact tip alters the water content in the area where the one or more fractional protrusion is compressed. More specifically, in the area where the fractional protrusion is compressed the water contact is decreased and the water escapes to other regions of the tissue that are not compressed. Accordingly, the surrounding tissue that is not compressed by the fractional protrusion may experience at least some expansion.

Referring now to FIG. 7A, in one embodiment, the protrusion 701 of the fractional contact tip 700 has a first width (w₁) measuring about 4 mm, a second width (w₂) measuring about 2 mm, and a depth (d) measuring about 1.5 mm. The temperature of the protrusion is controlled to measure about 5° C. and the initial temperature of the subject's tissue (i.e., the epidermis 110, the dermis 120, the fat 130 and the muscle 140) prior to contact with the 5° C. protrusion 701 of the fractional contact tip 700 measures about 37° C.

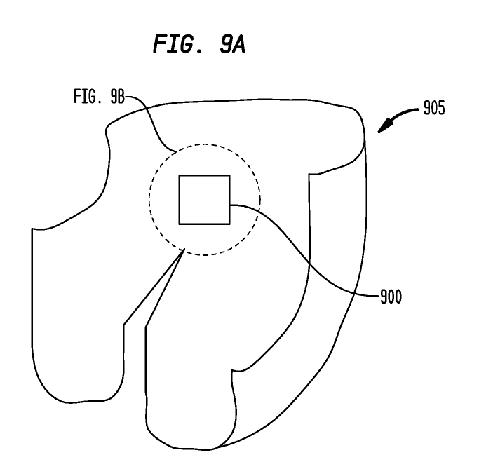

FIG. 8A shows a plot of the change in tissue temperature over time when the tissue, which starts at a temperature measuring about 37° C., is exposed to an ordinary contact tip (i.e., the non fractional contact tip 800 shown in FIG. 7B) having a temperature of 5° C. FIG. 8A also shows the change in tissue temperature over time when the tissue, which starts at a temperature measuring about 37° C., is exposed to a fractional contact tip (e.g., the fractional contact tip 700 with protrusion 701 shown in FIG. 7A) having a temperature of 5° C. FIG. 8A shows that the temperature of the subject's tissue drops faster when exposed to a fractional contact tip 700 (shown in FIG. 7A) as compared to when the subject's tissue is exposed to an ordinary contact tip (i.e., non fractional contact tip 800 shown in FIG. 7B) when both the contact tips 700, 800 are held at 5° C. and the subject's tissue is at a starting temperature of 37° C. Referring still to FIG. 8A, the location where the tissue temperature is measured after application by the ordinary contact tip 800 and the fractional contact tip 700 is about 1 mm below the dermis/subcutaneous junction. However, the locus of the dermis/subcutaneous junction deepens upon exposure to the fractional contact tip 700, which is caused by the protrusion 701 of the fractional contact tip 700 pushing against the dermis/subcutaneous junction and this locus deepens by about 0.1 mm relative to the ordinary contact tip.

FIG. 8B illustrates that the benefit of fractional application of compression and/or pressure in terms of the depths accessible by employing a fractional contact tip outweighs the negatives associated with the decrease of diffusivity due to the displacement of water caused by the application of fractionally applied pressure and/or compression. FIG. 8B has an x-axis of time measured in seconds plotted against a y-axis of temperature measured in ° C. Two curves measure the change in temperature as a function of a time for a fractional contact tip (e.g., the fractional contact tip 700 having the protrusion 701) applied to a fixed water content and applied to a variable water content. FIG. 8B illustrates that the water content (whether fixed or variable) provides a minimal impact on the temperature change over time on a subject's tissue upon exposure to a fractional contact tip.

When cycling cooling with optical energy heating a light based fractional treatment can provide the optical energy heating. In currently existing fractional light based technology, fractional columns have generally been made deeper by applying more energy, which has other ramifications, including cost of the device, the time required for treatment to be completed, the application of more power to the tissue, which can result in more damage to the tissue and the diffusion of energy within the tissue. A desired depth that can be achieved using less energy will allow a device to use less energy, a less costly photo thermal damage source and/or the application of less energy per volume of tissue. When cycling cooling with optical energy heating, applying pressure when conducting the optical energy heating portion of the cycle can achieve a deeper depth of treatment with less energy. In addition, the time required to achieve heating is reduced. In many current fractional treatment applications, the depth of penetration of the fractional columns into the tissue is believed to be important to the effectiveness of the treatment. In applications where depth of penetration into human skin is important, a deeper column capable of reaching deeper into the dermis, hypodermis, and/or subcutaneous fat tissue will result in greater effectiveness of the treatment.

Depending on the treatment goals the target depth can be the dermis, fat, and/or the muscle. Both fat tissue and muscle tissue are relatively deep structures that may by suitably treated by fractional cooling, by cycling fractional cooling and heating, and by combining fractional cooling and fractional heating.

A thermal element (e.g., a thermal control element) can be employed to solely cool, solely heat, cycle cooling and heating, and/or combine cooling and heating to an external surface of a subject's body such as, for example, the skin. The thermal element can provide a fractional application that combines cooling and heating with region(s) of cooled treatment adjacent to regions of non treatment and adjacent to region(s) of heated treatment.

In some embodiments, a region of cooling is adjacent to a region of heating. For example, in one embodiment the thermal element includes, for example, concentric circles with an internal circle providing thermal control that differs from the thermal control provided by a circle external to the internal circle. In this way, inside circles alternately provide heating and cooling (e.g., an inner circle provides heating and an outer circle external to the inner circle provides cooling). Such variance in thermal control can repeat for additional external and/or internal circles providing thermal control.

In another embodiment, a thermal element provides a region of cooling adjacent to a region of heating having a different pattern such as, for example, a line of heating adjacent to a line of cooling, or an array of cooling elements adjacent to an array of heating elements in a pattern similar to, for example, a checkerboard.

When employing a thermal element it is desirable to avoid suppression of an immune system through significant hypothermia. A strategy that combines cooling and heating can avoid significant hypothermal by sizing the sub-elements of each thermal element from about 3 mm (minimum) to about 6 inches. At least one aspect of the size of the sub-element of a thermal element should approximate the depth penetration that you hope to achieve by employing the thermal element. The smallest characteristic dimension of a sub-element of a thermal element e.g. where the thermal element is a line, a square, a circle, or a rectangle, for example, the depth of penetration of that element in the tissue will be no smaller than that smallest characteristic dimension of the thermal element. More specifically, where the thermal element is a relatively long rectangle the width of the relatively long rectangle is the characteristic dimension of the thermal element. Where the thermal element is a circle the smallest characteristic dimension is the diameter of the circle. Where the thermal element is an ellipse the smallest characteristic dimension is the minor axis. Whether the thermal element is a square the smallest characteristic dimension is the minor side of the square.

In some embodiments, a device applies thermal elements for cooling, heating, cycled cooling and heating, and/or a combination of cooling and heating applied to the external surface of a subject's body upon the application of pressure and/or compression to the device. The device can be employed for a long period of time to one or more areas of a subject's body. Suitable devices that apply pressure and/or compression can be, for example furniture, garments, or other appliances that may be located in a region adjacent the subject's skin. In some embodiments, where, for example, the device is a piece of furniture such as a chair, a sofa, and/or a mattress the weight of the subject provides compression to the subjects skin. In other embodiments, the device is a garment and the elasticity of the garment provides compression and/or pressure to the subject's skin. The garment can exert adjustable pressure such as is possible with, for example, an adjustable garment akin to a blood pressure cuff. A garment can be made of a material that provides at least some elasticity, such as, for example, a patch, an adjustable cuff, pants, shirts, dresses, undergarments made of a material having elasticity such as a lycra material. Suitable wearable garments can be made from a relatively inflexible material such as, for example, wood, plastic, metal and paperboard. In other embodiments, all or a portion of the device is inflated to provide pressure to the subject's skin. In other embodiments, all or a portion of the device is under vacuum, which applies pressure and/or compression to the subject's skin.

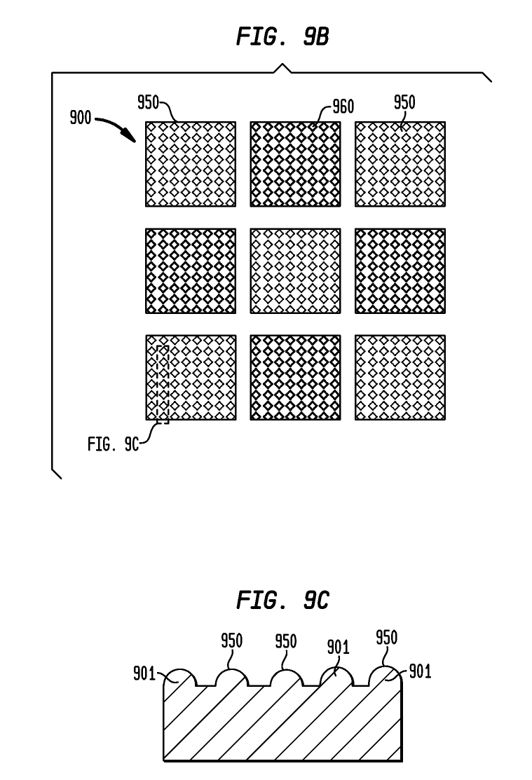

FIG. 9A shows a diagram of a piece of furniture, i.e., a chair 905 having one or more fractional contact tips 900. Referring to FIGS. 9B and 9C, the fractional contact tip 900 includes a combination of protrusions 901 for cooling 960 (e.g., a group of cooling protrusions) adjacent to protrusions 901 for heating 950 (e.g., a group of heating protrusions). The cooled and/or heated protrusions are applied to the external surface of a subject's body upon the application of pressure and/or compression to the device. Where the device is a piece of furniture (i.e., a chair 905), the body weight of the subject provides the pressure and/or compression of the subject's skin to the fractional contact tip 900 within the chair 905. In some embodiments, the protrusions 901 within the fractional contact tips 900 are activated (i.e., set off) by the pressure and/or compression of the weight of a person sitting on the furniture. In embodiments where the protrusions within the fractional contact tips are contained within a garment the compressive fit of the garment and/or the vacuum or other exertion of pressure of the fractional contact tips via the garment can be activated (i.e., set off) by the fit of the garment (i.e., elastic fit, compressive fit, and/or the vacuum fit of the garment).

In one embodiment, the surface temperature that contacts the surface of the subject's skin by the cooling 960 protrusions have cold temperatures that range from about −5° C. to about 20° C., or about 0° C. In one embodiment, the surface temperature that contacts the surface of the subject's skin by the heating 950 protrusions have heated temperatures that range from about 37° C. to about 45° C., or about 40° C.

Such devices may be used to provide compression and/or pressure to the external surface of a subject's skin for a relatively long period of time. Such devices provide compression via the weight of the subject, the elasticity of the garment, inflation of the device against the skin of the subject, and/or vacuum pressure, for example. Without being bound to any single theory, it is believed that a key to success is that heat is adjacent cooling. In order to avoid and/or lessen the risk of damage to blood heating the heated area should be limited and the cooled area can be relatively larger than the heated area. More specifically, the area of heating divided by the area of cooling can be represented as Formula 3:

$$\frac{Δ heating}{Δ cooling} \qquad ( Formula 3 )$$

One can adjust the surface area to be heated and to be cooled by the device in order to prevent the surface area of the subject's total body from over heating or over cooling. In addition, the surface area that is heated can be altered so that it is varied from the surface area that is cooled, in this way the treatment of the subject can be altered to target a desired treatment level and/or a desired outcome.

In order to avoid and/or lessen the risk of damage to the subject's body, the total heat flux put into the body should be substantially equal to the total heat flux removed via cooling, which is represented by Formula 4.

$$\frac{{S}_{heating}}{{S}_{cooling}} = \frac{Δ {T}_{cooling}}{Δ {T}_{heating}} \qquad ( Formula 4 )$$

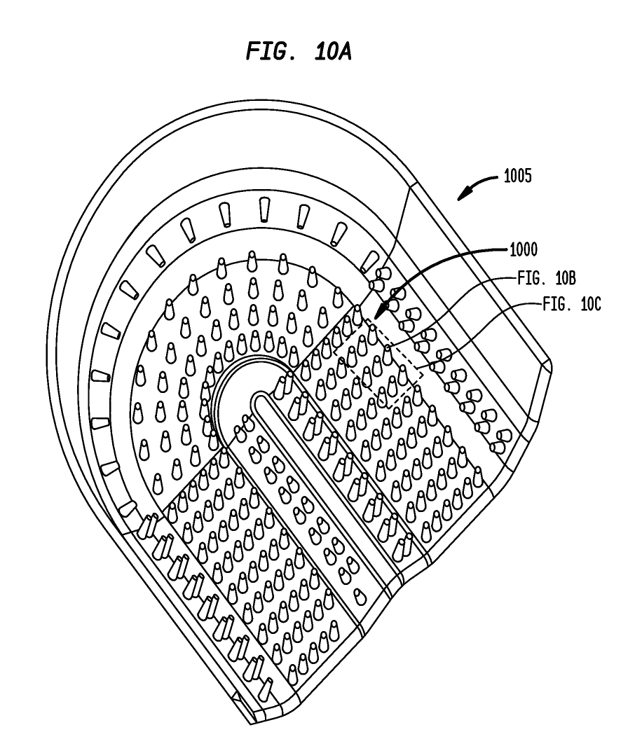

FIG. 10A shows an exemplary piece of furniture, specifically, the seat of a chair 1005 having one or more fractional contact tips 1000. The seat of the chair 1005 shown in FIG. 10A can provide radiation via heating and/or cooling. The seat of the chair 1005 shown in FIG. 10A can be described as a needle seat. The temperature controlled needles can allow fractional treatment of conditions such as, for example, cellulite. The protrusions of the fractional contact tips may be thermally controlled by being pre-chilled and/or pre-heated or actively temperature controlled by, for example, thermo electrical cooling (i.e., TEC), circulating temperature controlled fluid and/or electromagnetic energy (e.g., light energy or microwave energy). The seat of the chair 1005 can be made from any of a number of material(s) including materials transparent to electromagnetic energy such that electromagnetic energy sources can be placed adjacent the seat (e.g., on one or more sides of the seat of the chair 1005). Suitable devices can be employed in accordance with the device described in FIGS. 10A, 10B, and 10C, for example, and include various other types of furniture (e.g., mattresses and/or sofas) and garments (e.g., pants, shirts, dresses, undergarments, and/or blood pressure cuffs) for example that are employed as applicators that are confined to an anatomical area of a subject and enable application for a relatively long period of time.

FIG. 10B shows an embodiment of fractional contact tips 1000 in which protrusions are solid. The solid protrusions 1001 heat and/or cool the external surface of a subject's skin by, for example, direct contact of the solid protrusions 1001 with the external surface of the skin.

FIG. 10C shows an embodiment of fractional contact tips 1000 in which protrusions 1001 are not solid, but rather, are at least partially hollow. The at least partially hollow protrusions 1001 can have internal jets. The external surface of a subject's skin can be convectively cooled by direct impingement onto the skin with a temperature controlled (i.e., heated and/or cooled) fluid. The temperature controlled fluid can be a liquid, vapor and/or dry ice particles. The temperature controlled fluid can convectively cool and/or heat the subject's skin via external means by applying the fluid to the external skin surface from the internal jets in the protrusions 1001 in the fractional contact tips 1000.

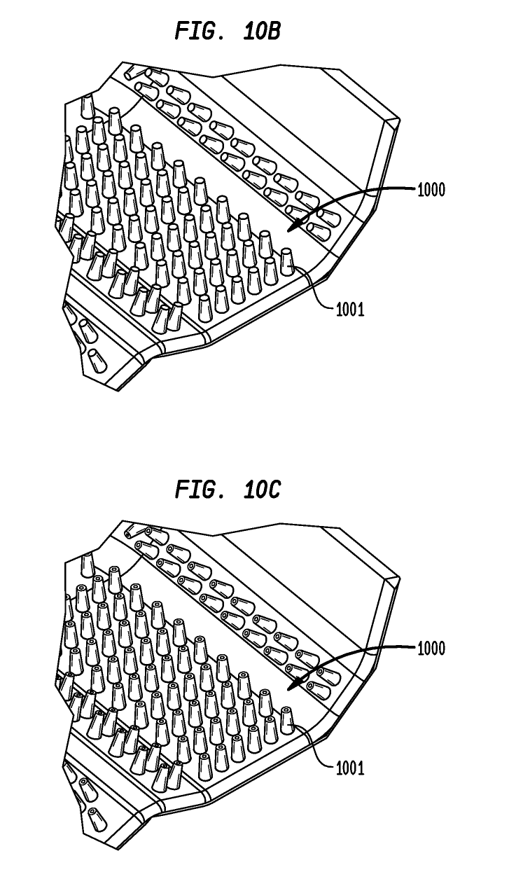

FIG. 11 shows a cup 1107 with one or more protrusions 1101 on an inner surface of the cup 1107. Instead of pushing metal into the external surface of the subject's skin, the cup 1107 is employed to bring the subject's skin into the cavity 1177 of the cup 1107 via vacuum 1199. The cup 1107 can be thermally conductive. The protrusions 1101 can be employed for fractional cooling by employing vacuum 1199 to put negative pressure relative to the skin surface to pull the subject's skin tightly against the temperature controlled protrusions 1101, which cools at least a portion of the subject's skin. A thermal agent 1133 can circulate within the walls of the cup 1107 to heat and/or cool all or a portion of the cup 1107 including, for example, the protrusions 1101 disposed on the inside surface of the cup 1107.

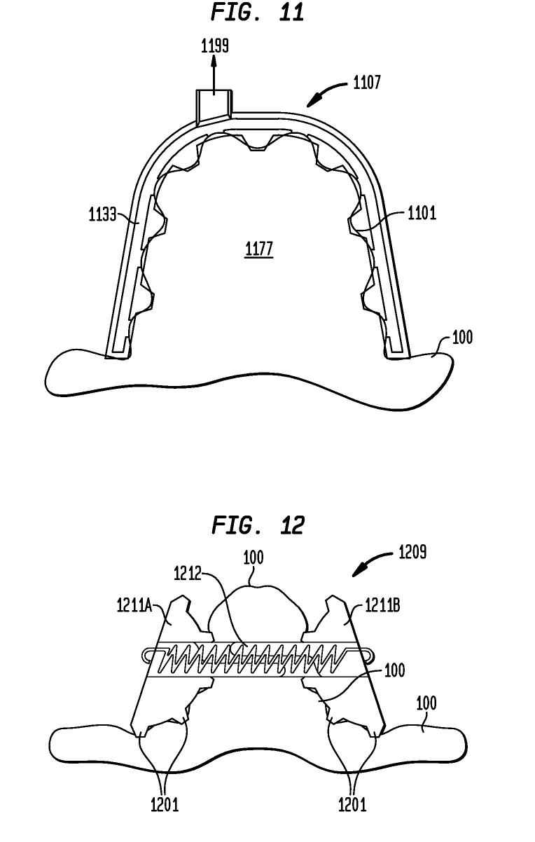

FIG. 12 shows a device 1209 that works like a vice having a first side 1211A and a second side 1211B each side having protrusions 1201 on a surface of each side 1211A, 1211B. The protrusions 1201 contact the subject's skin 100 when it is located in between the two sides 1211A, 1211B when the vice is activated. The device 1209 can alternatively be described as a spring clamp with protrusions 1201 located on the sides 1211A, 1211B of the clamp. The protrusions 1201 can compress the skin 100. The protrusions 1201 can be temperature controlled to cool and/or heat the skin 100 held therebetween. A spring 1212 can be disposed between the sides 1211A and 1211B. The spring 1212 can be, for example, a simple direct acting mechanical device, a pneumatic actuator, a hydraulic actuator, a piston, and/or a bladder diaphragm. The spring 1212 together with the two sides 1211A, 1211B compress and/or provide compression on the skin 100 such that the protrusions 1201 can fractionally treat the skin 100 compressed therein.

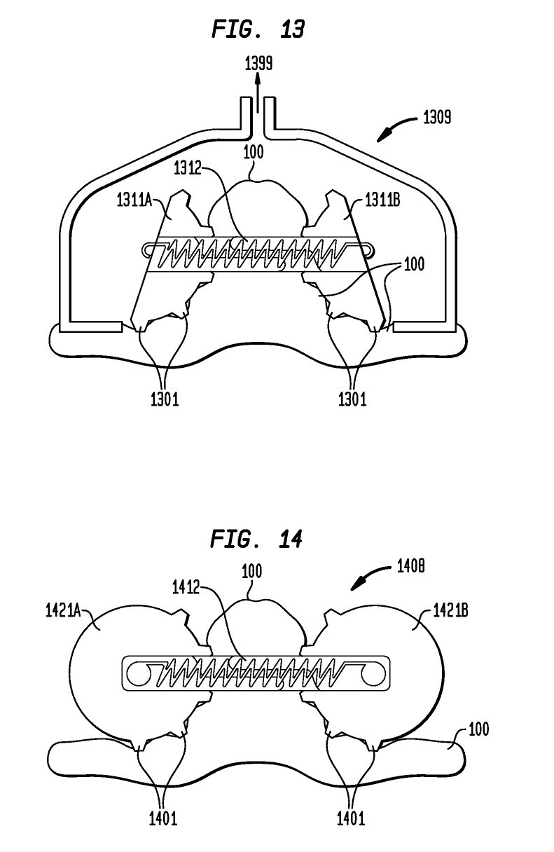

FIG. 13 shows a device 1309 that works like a vice 1209 described in FIG. 12, the device 1309 having a first side 1311A and a second side 1311B each side having protrusions 1301 on a surface of each side 1311A, 1311B. The protrusions 1301 contact the subject's skin 100 when it is located in between the two sides 1311A, 1311B when the vice is activated. The device 1309 can also be described as a spring clamp with protrusions 1301 located on the sides 1311A, 1311B of the clamp. The protrusions 1301 can compress the skin 100. The protrusions 1301 can be temperature controlled to cool and/or heat the skin 100 held therebetween. A spring 1312 can be disposed between the sides 1311A and 1311B. The spring 1312 can be, for example, a simple direct acting mechanical device, a pneumatic actuator, a hydraulic actuator, a piston, and/or a bladder diaphragm. Vacuum 1399 can be employed to assist in providing contact between the subject's skin 100 and the protrusions 1301. By employing vacuum 1399, the pressure exerted on the skin 100 by the protrusions 1301 is increased. FIG. 13 depicts vacuum 1399 applied from a top portion of the device 1309, however, vacuum 1399 can be applied from other regions of the device 1309 such as, for example, one or more sides for example in the region of the first side 1311A or the in the region of the second side 1311B.

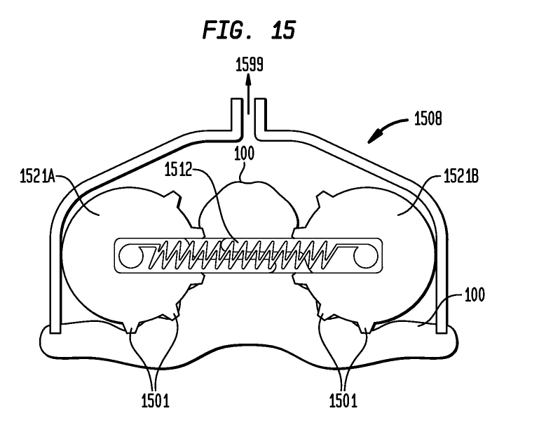

FIG. 14 shows a device 1408 that includes at least two opposing rollers, the first roller 1421A and the second roller 1421B each have one or more protrusions 1401 disposed on the external surface of each of the opposing rollers 1421A, 1421B. The subject's skin 100 is located between the opposing rollers 1421A, 1421B. The protrusions 1401 located on the external surface of the rollers 1421A, 1421B can compress at least a portion of the skin 100. A spring 1412 can be disposed between the rollers 1421A and 1421B. The spring 1412 can be, for example, a simple direct acting mechanical device, a pneumatic actuator, a hydraulic actuator, a piston, and/or a bladder diaphragm. The spring 1412 can provide tension between the first roller 1421A and the second roller 1421B to provide pressure and/or compression of the protrusions 1401 against the skin 100. The protrusions 1401 can be temperature controlled to cool and/or heat the skin 100 held therebetween. In this way, the protrusions 1401 can provide temperature controlled fractional treatment, for example, fractional cooling. The opposing rollers 1421A, 1421B can gather and compress the skin 100 against the protrusions 1401 that are disposed on the external surface of the rollers 1421A, 1421B. The opposing rollers 1421A, 1421B can knead the skin 100 held therebetween to compress and/or apply pressure to the skin 100 via the one or more protrusions 1401 disposed on the surface of the opposing rollers 1421A, 1421B.

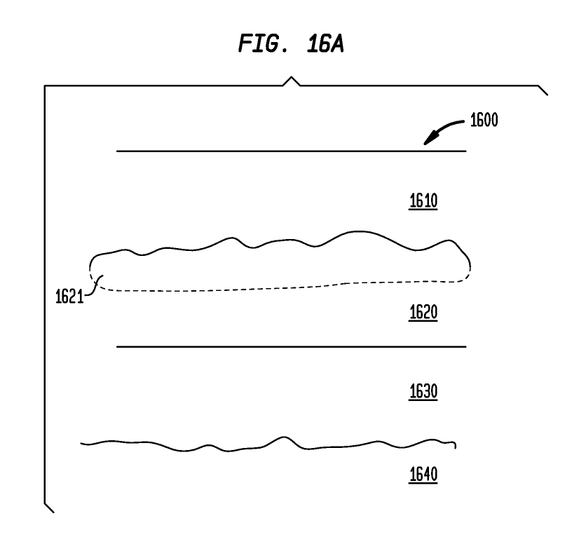

FIG. 15 shows a device 1508 that includes at least two opposing rollers, the first roller 1521A and the second roller 1521B each have one or more protrusions 1501 disposed on the external surface of each of the opposing rollers 1521A, 1521B. The subject's skin 100 is located between the opposing rollers 1521A, 1521B. The protrusions 1501 located on the external surface of the rollers 1521A, 1521B can compress at least a portion of the skin 100. A spring 1512 can be disposed between the rollers 1521A and 1521B. The spring 1512 can be, for example, a simple direct acting mechanical device, a pneumatic actuator, a hydraulic actuator, a piston, and/or a bladder diaphragm. The spring 1512 can provide tension between the first roller 1521A and the second roller 1521B to provide pressure and/or compression of the protrusions 1501 against the skin 100. The protrusions 1501 can be temperature controlled to cool and/or heat the skin 100 held therebetween. In this way, the protrusions 1501 can provide temperature controlled fractional treatment, for example, fractional cooling. The opposing rollers 1521A, 1521B can gather and compress the skin 100 against the protrusions 1501 that are disposed on the external surface of the rollers 1521A, 1521B. Vacuum 1599 can be employed to assist in providing contact between the subject's skin 100 and the protrusions 1501. By employing vacuum 1599 the pressure exerted on the skin 100 by the protrusions 1501 may be increased. FIG. 15 depicts vacuum 1599 applied from a top portion of the device 1508, however, vacuum 1599 can be applied from other regions of the device 1508 such as, for example, one or more sides for example in the region of the first roller 1521A or the in the region of the second roller 1521B.

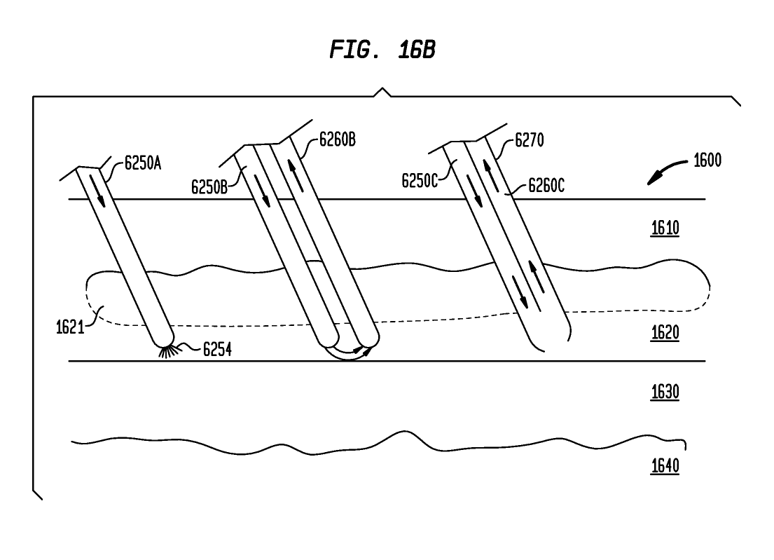

Treatment of tissue (e.g., fat tissue) with cooling can be employed for body sculpting and/or fat tissue removal. FIG. 16A shows a cross section of human tissue 1600 that includes a layer of skin 1610, a layer of fat 1620 below the layer of skin, a layer of muscle 1630 below the fat layer 1620 and, optionally in some embodiments another layer of fat 1640 below the layer of muscle 1630.

Suitable approaches to cooling fat tissue can include applying cooling (e.g., a cooling panel, a cooling agent, and/or a cooling device) to the skin surface for a period of time that causes the temperature of the skin and the subcutaneous regions below the skin to drop relatively gradually. Drawbacks associated with employing an external approach to cooling include a limit to the subcutaneous tissue depth that may be achieved. For example, when cooling is approached from an external surface of the subject's body by employing, for example, an ordinary non fractional contact tip as discussed in relation to FIG. 7B, it is sometimes not possible to go deeper than the superficial layer 1621 of the fat layer 1620 under the skin 1610. The superficial layer 1621 has a depth that ranges from about 1 mm to about 5 mm, for example, the portions of the fat layer 1620 below the superficial layer 1621 range from about 5 mm to about 25 mm. Going deeper than the superficial layer 1621 of the fat layer 1620 by external cooling means would take a relatively long period of time (e.g., from about 10 minutes to about 120 minutes, or about 60 minutes) and could lead to over cooling of the skin and/or over cooling of the superficial layer 1621 of the fat layer 1620 and/or over cooling of the subject's body. Exposing tissue to over cooling can lead to irreversible damage (e.g., irreversible skin damage) and likewise the subcutaneous layer can be burned.

Another means of cooling all or a portion of a fat layer (e.g., fat layer(s) 1620 or 1640) is delivering cooling directly into the fat layer (e.g., fat layer(s) 1620 or 1640). To provide controllable and even cooling of human fat one can use different approaches including delivery of cooling agents or delivery of cooling devices directly into one or more fatty layer of tissue.

In one embodiment, cooling is achieved by delivering a cooling agent into the fat layer 1620, 1640, via, for example, direct delivery. In this way, a cooling agent (e.g., a cooling fluid or a cooling crystal) is directly exposed to human fat tissue. In one embodiment, the cooling agent is directly delivered into the fat tissue by, for example, injection and/or by introducing a channel into the tissue to enable delivery of the cooling agent thereto. Such methods can enable controlled cooling of subcutaneous fat by injecting specified amount of cooling agent (e.g., cooling fluid and/or cooling crystal) with predetermined temperature, directly into the fatty tissue to be treated. The predetermined temperature of the cooling agent can be in the range of from about −20° C. to about 20° C., from about −5° C. to about 15° C., or from about −5° C. to about 5° C. The temperature of the phase transition of a cooling crystal should be in the same range, in the range of from about −20° C. to about 20° C., from about −5° C. to about 15° C., or from about −5° C. to about 5° C. Ice provides a particularly suitable cooling crystal, because the phase transition temperature is 0° C. and ice has good biocompatibility with the subject's tissue.

In one embodiment, a cooling agent is delivered directly to the layer of fat tissue by injection. Injection of a cooling agent can be accomplished by employing a needle and/or a cannula, which is introduced into the tissue (e.g., into the fat tissue 1620 and/or 1640). Optionally, the cannula may be a delivery and/or irrigation cannula. In some embodiments, one or more incisions are made in the skin surface 1610 prior to inserting the needle and/or the cannula into the desired region of the tissue. Thereafter, the cannula is moved to a desired depth within the subject's tissue 1600, e.g., the fat layer 1620 or 1640.

In another embodiment, a hole and/or a channel is cut into the tissue. Suitable channels may be, for example, an ablative channel (or a series of fractional ablative channels) created with a laser such as the fractional Lux 2940 available from Palomar Medical Technologies. The skin 1610 and/or the fat layer (1620 and/or 1640) below the skin 1610 can be accessed through one or more channel to deliver cooling agent(s) to the fat layer using mechanical force (e.g., pressure). Pressure can be achieved by injecting one or more cooling agent into the fat layer 1620 and/or 1640 by, for example, high pressure injection of the cooling agents into the channel disposed in the tissue.

Any of a number of cooling agents may be employed in accordance with the methods and devices disclosed herein. In one embodiment, the cooling agent is a slurry of water with suspended ice particles. The slurry can be made from about 1% ice to about 99% ice, or from about 10% ice to about 85% ice, or from about 20% ice to about 75% ice, or from about 25% ice to about 50% ice or about 35% ice. The amount (e.g., the percentage) of ice in the slurry of the cooling agent depends on the target tissue (e.g., the amount of ice in the slurry will depend on the size of the channel and/or the volume of the fat to be treated thus the target tissue to be treated melts the ice of the slurry introduced therein). Generally it is desirable to have a greater amount of ice in the slurry to keep the temperature of the area being treated in the range of from about 0° C. to about 20° C.

A certain volume of tissue is required to melt ice of the slurry and if there is enough ice in the slurry to enable the tissue to fall below 20° C. then crystals form in the fatty tissue and/or the fatty cells in the fat tissue. Crystal formation in the fatty cells is desired to enable fat cell destruction. Once the slurry is disposed in the subject's body (e.g., in a region of fat tissue) and the slurry cools the targeted tissue (e.g., causes apoptosis) the subject's body can process the water slurry and the water via absorption. Thus, it may not be necessary to evacuate the water that remains after the slurry cools the subject's body, rather the subject's body processes the water. The ice of the slurry can range in size from about 1 micron per crystal to about 3 mm per crystal, or about 100 microns per crystals.

In an embodiment when an ice slurry is employed as a cooling fluid, the temperature of the slurry (Tₛ) is about 0° C. In order to provide even cooling of a region of tissue (e.g., fatty tissue) to be treated, the operation field of the tissue has the dimensions L×W×D (where L is the length of the tissue to be treated, W is the width of the tissue to be treated and D is the depth of the tissue to be treated) from an initial temperature (Tᵢ) of from about 30° C. to about 36° C. to a treatment temperature Tₜ the amount of ice slurry that should be employed is represented by G assuming a slurry that is 100% ice:

G=L*W*D*C_{f}*Den*(Tᵢ−Tₜ)/Lₕ+L*W*D*C_{f}*Den*(Tᵢ−Tₜ)/(Cₛ*Denₛ*(Tₜ−Tₛ) (Formula 5)

where:

C_{f} is fat heat capacity, J/Kg*Celsius

Den is fat density, Kg/m³

Lₕ is latent heat of ice slurry, J/kg,

Cₛ is water heat capacity, J/Kg*m³

Denₛ is water density, Kg/m³.

In illustrative embodiment No. 1:

the tissue to be treated (e.g., the operation field) is the equivalent of the human abdomen area having dimensions of L=0.25 m, W=0.40 m, and D=0.01 m,

The fat heat capacity C_{f}=2300 J/Kg*Celsius,

The fat density Den=850 kg/m³,

The latent heat of the ice slurry is Lₕ=334000 J/kg,

The water heat capacity Cs=4200 J/Kg*m³,

The water density is Denₛ=1000 Kg/m³.

Thus, in order to provide adiabatic cooling of fat of dimensions provided in illustrative embodiment No. 1 from an initial temperature of the tissue to be treated Tᵢ=30° C. to a treated temperature Tₜ=10° C. one should expend about G˜0.005 Kg of ice slurry, where the ice slurry is 100% ice.

In another embodiment, an evaporative cooling agent is employed to cool at least a portion of tissue (e.g., fat tissue) via sublimation. One suitable evaporative cooling agent includes CO₂ crystals (i.e., microcrystals). The temperature of the CO₂ crystals is about −78.5° C. In one embodiment, such evaporative crystals are injected into the subject's tissue and the evaporative crystals start to sublimate and/or melt and turn into a gas. Once delivered into the body, the size of the CO₂ crystals must be small enough in size to avoid over damaging the tissue (over damaged tissue features at least some crystallization of the water and/or crystallization of fat cells in the tissue caused by, e.g., contact with CO₂ crystals). Thus, the evaporative crystals must be sized such that they limit the cooling capacity of the tissue to which they are exposed thereby to avoid over cooled and/or over damaged tissue. The evaporative crystals must be sized such they start out large enough to make it to the target region of the body (i.e., the fat tissue), because some melting and/or sublimation occurs during the time period in which the CO₂ crystals travel from one location to the area for treatment. The crystal size can range from 1 about micron to about 100 microns per crystal. The evaporative crystals need to be handled and sized so that they do not over sublimate and/or over melt such that they have a suitable size to treat the fat layer once the evaporative crystals reach the fat layer. A certain volume of tissue is required to melt and/or sublimate the CO₂ crystal and if there is enough CO₂ crystal to enable the tissue to fall below 20° C. then crystals form in the fatty tissue and/or the fatty cells in the fat tissue. Crystal formation in the fatty cells is desired to enable fat cell destruction.

Sublimation and/or melting create gas through evaporation from the crystal state of the CO₂ crystals. One concern is that the CO₂ gas can create a pressure build up within the subject's body that risks damaging tissue due to exposure to the pressure caused by the gas. The CO₂ gas build up can be controlled by, for example, employing one or more cannulae, one or more multichannel cannulae, channels drilled into the tissue, and/or valves in cannulae. In one embodiment, the gas created by CO₂ crystal evaporation can exit the subject's body via the point of entry by which the CO₂ crystals were introduced into the subject's body. In another embodiment, a separate exit port is created in the tissue to enable removal of the gas from the subject's body.

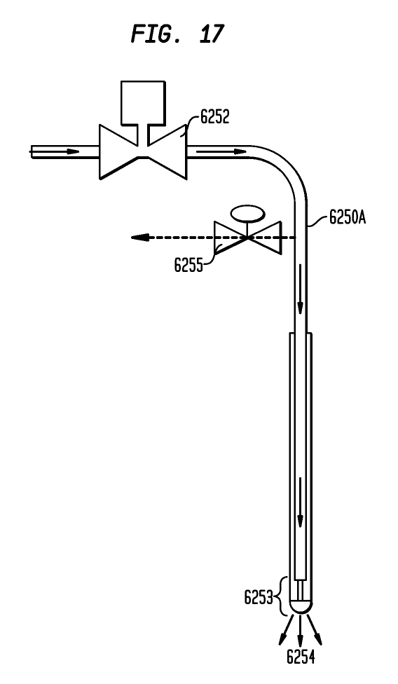

FIGS. 16B and 17 show a cannula 6250A that delivers a cooling agent (e.g., a cooling liquid and/or one or more CO₂ crystals) to human tissue 1600 to a depth within the fat layer 1620. As discussed herein, suitable cannula can be a vessel containing cooling agent for cooling a target treatment volume. The cooling agent is supplied under pressure through a modulating valve 6252 and is delivered into the tissue from an opening 6254 in the outlet side of the cannula. The cooling agent may be a chilled liquid, a gas or a slurry such as an ice slurry, for example. The cooling agent might also be CO₂ gas which undergoes a phase change as it passes through a restrictor device (orifice or nozzle) which is optionally located in the modulator valve 6252 or in the cannula, for example. FIG. 17 shows the restrictor 6253 located substantially at the tip of the cannula 6250A. In the case of CO₂, the high pressure gas would sublimate to dry ice particles at the outlet 6254 located downstream of the restrictor 6253. The restrictor outlet and/or restrictor device is a metering device that can properly meter flow of cooling agent into the treatment volume. The restrictor device may be, for example, an orifice, a nozzle, or a capillary tube. The flow rate and particle size could be controlled to prevent over cooling of tissue. Additionally a pressure relief mechanism such as a pressure regulating valve 6255 may be provided to prevent over pressurizing the treatment volume. The modulating valve 6252 can be a control valve with an actuator that is employed to control the flow of the cooling agent. In some embodiments, the control valve with the actuator is controlled by a sensor contained in a feedback loop. The sensor may sense any of a number of data such as, for example, one or more of temperature of cooling agent, temperature of tissue, pressure, position of cannula, and/or the treatment volume.

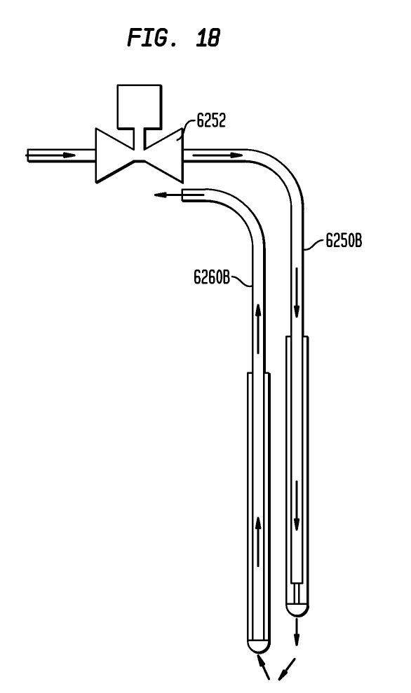

FIGS. 16B and 18 also show a cannula 6250B that delivers a cooling agent (e.g., a cooling liquid and/or one or more CO₂ crystals) to human tissue 1600 to a depth within the fat layer 1620. Another cannula 6260B is employed to aspirate liquid and/or gas (e.g., CO₂ gas) from the subject's body. FIG. 18 shows that the cannula 6250B is similar to the cannula 6250A for delivering cooling agent discussed in relation to FIG. 17, however a “vent” cannula 6260B is added to allow excess coolant (e.g., in liquid and/or in gas form) to be removed from the treatment volume.

FIG. 16B also shows a multi lumen cannula 6270 (e.g., a dual lumen cannula) that includes a delivery portion 6250C and an aspiration portion 6260C. The multi lumen cannula 6270 can deliver a cooling agent (e.g., a cooling liquid and/or one or more CO₂ crystals) to human tissue 1600 at a depth within the fat layer 1620 through the delivery portion 6250C. Another portion 6260C of the multi lumen cannula 6270 can be employed to aspirate liquid and/or gas (e.g., CO₂ gas) from the subject's body. In some embodiments, the multi lumen cannula is adapted to deliver medication through the delivery portion 6250C or, optionally, through another portion of the multi lumen cannula (not shown). In some embodiments, immediately after exiting the delivery cannula and/or the delivery portion the cooling agent returns back via the aspiration cannula.

To provide an even cooling effect, the delivery and/or aspiration system(s) may be equipped with a thermal sensor embedded in the region of the cannula (e.g., adjacent the location where the cooling agent exits the delivery cannula) and/or a thermal sensor embedded in the region of the aspiration cannula (e.g., at the point of entrance into the aspiration cannula).

In some embodiments, the delivery cannula is equipped with controllable valve that provides a predetermined amount of cooling agent (e.g., cooling liquid and/or frozen particles such as CO₂ crystals, for example). The predetermined amount of cooling agent may be calculated after temperature measurement by, for example, embedded thermal sensor(s). In another embodiment, a thermal sensor, a thermal sensor signal, and/or a valve employed in conjunction therewith could be used to control the temperature and/or the pressure of the cooling fluid being delivered by the delivery cannula and/or the delivery portion. In another embodiment, the delivery cannula is equipped with position sensor that provides a substantially even distribution of the cooling agent in the subject's tissue. The signal of a position sensor may be used to control the amount of cooling fluid delivered though the delivery cannula and/or the delivery portion. In some embodiments, the signal of a position sensor and a valve are employed together to control the amount of cooling fluid delivered through the delivery cannula and/or the delivery portion.

Suitable cannulae may be cooled directly (e.g., without a cooling agent) by employing an attached TEC or other heat exchanger that is coupled to a suitable cooling system, such as, for example, a refrigeration device. Thermal conduction of the cannula may be enhanced by integrating an internal heatpipe (e.g., an evaporator).

In another embodiment, a cooling device is exposed to the fatty tissue to be treated. For example, a portion of a cooling device is cold and/or has a cold tip. The cooling device is cooled by 1) circulating liquid and/or gas within the cooling device and/or 2) thermoelectrically cooling the cooling device. This cooling device and/or cooling device tip could be used to generate a hypothermic zone in the fatty tissue.

In one embodiment, the cooling device is a cannula and/or a cannula tip that is sized and shaped to enable the cannula to traverse within the tissue. In another embodiment, the cooling device is a rod having a tip shaped to enable the rod to traverse within the tissue. In some embodiments, a portion of the subject's tissue is cut to enable insertion and placement of the cooling device and/or the cooling device tip in the area to be treated. The cold zone created by the cooling device can be controlled by the number of strokes in the zone of treatment. Once the treatment zone is brought to the desired temperature, the cooling device can be removed from the treatment area.

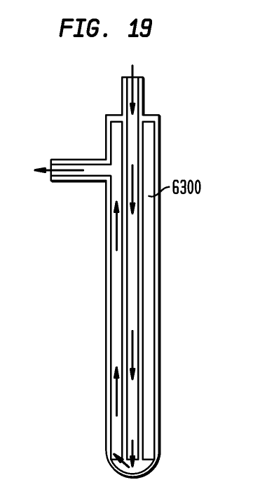

In one embodiment, referring to FIG. 19, an advantage of using the cooling device is that you don't need to inject any agent into the tissue to be treated, rather during the treatment you provide contact of the tissue with the cold device and/or the cold tip of the device and evacuation of the tissue is not necessary. Such “closed loop” cannulae 6300 circulate a volume of cooling agent therein to provide the desired level of cooling. Metering devices may be employed to properly meter the flow of cooling agent within the cannula 6300. Suitable cooling agents that may be employed to cool the cooling device include, for example, liquid, vapor, liquid nitrogen, freon, slurry (e.g., having from about 1% to about 99% of frozen particles (e.g., ice) suspended in a liquid), cold water, and/or CO₂ sublimation. In some embodiments, cooling agent(s) are contained in a multi lumen cannula 6300 and cooling action is promoted through the walls of the cannula 6300 without contact between the cooling agent(s) contained in the cannula and the tissue, in this way, the cooling agent is not directly delivered to the tissue, rather it cycles within the cannula 6300 (e.g., the multi lumen cannula) so as to cool the tissue in contact with all or a portion of the cannula 6300 (e.g., the multi lumen cannula).

Such a cooling device can be employed to create a cooling zone, and the cooling zone leads to apoptosis of fat cells thereby destroying fatty tissue. It is possible to leave the treated tissue (e.g., the fatty tissue in the cooled zone) in place within the body of the subject. In this way, the subject's body will metabolize the damaged and/or dead fat cells and additional lipids that were damaged by the cooling process.

In another embodiment, the cooled zone of tissue can be treated by thermo cycling with, for example, warming (e.g., treated with a warm cannula, treated via laser light, treated with electrical energy, and/or treated with ultrasound energy). Thermo cycling by heating a previously cooled zone (e.g., immediately after creating the cooled zone) will generate liberated lipids (e.g., liquefied fat) which can be aspirated by, for example, suction, palpation, and/or aspiration. Once the cooled zone (e.g., the frozen fat) is heated the lipid can liberated thereby enabling immediate aspiration of the liberated lipid (e.g., via palpation through the hole or channel that entered the body to provide the cold treatment or by a separate hole or channel).

In some embodiments, the cold treatment plays a role in pain reduction such that the sensation of pain experienced by the subject can be diminished. In some embodiments, the cold treatment lessens blood circulation which can lessen and/or minimize and/or avoid bruising of the region of tissue that was treated. In some embodiments, the cooling agent (e.g., the cooling fluid) includes one or more vasoconstrictors. By employing vasoconstrictors, perfusion may be avoided and the residence time of the cooling fluid may be increased. In some embodiments, the cooling agent includes anesthetics, analgesics or other medications suited to decrease side effects and complications of treatment such as bruising and hemorrhaging, for example.

In accordance with the methods and devices disclosed herein, it is desirable to achieve a cold temperature of the treatment region of the fat layer that leads to dystrophy of the fatty tissue by killing fat cells of the tissue. In order to achieve dystrophy of fatty tissue the mechanism includes decreasing the temperature of the lipids in the fat cells below the temperature of fat cell crystallization, but above the temperature at which any water in the fat cells will crystallize, thus, the desired temperature range is from about 20° C. to about −5° C., or from below 20° C. to above −5° C.

In accordance with the methods and devices disclosed herein, any of the disclosed methods and devices can be combined with tumescent procedure(s) that are known in the medical arts. In one embodiment, a cooling liquid (e.g., an ice slurry) is delivered to the tissue via one or more delivery system typically used to deliver tumescence to a subject during a treatment.

In accordance with the methods and devices disclosed herein, any of the disclosed methods and devices for cooling tissue may be combined with a heating approach that is either internal to the body (using light energy, using ultrasound energy, using vibration energy, and/or using electrical energy) or external to the body (using light energy, using ultrasound energy, using RF energy, using heating via conduction and/or using magnetic energy such as induction as can be employed in ovens).

Thermal cycling as disclosed herein may be combined with coagulation of connective tissue. In such an application, a goal is reduction of the volume of adipose tissue and replacing the adipose tissue with more fibrotic tissue and/or smaller fatty cells. The sequential heating employed in thermal cycling could enhance blood flow and metabolism and have short term and/or long term impact on the subject's body. In accordance with the methods and devices disclosed herein, any of the disclosed methods and devices for thermo cycling may be have one or more sequences of cooling followed by heating. For example, in one embodiment, the thermo cycling sequences of cooling followed by heating may range from about 1 sequence to about 5 sequences.

### EQUIVALENTS

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described specifically herein. Such equivalents are intended to be encompassed in the scope of the appended claims.

### REFERENCES AND DEFINITIONS

The patent, scientific and medical publications referred to herein establish knowledge that was available to those of ordinary skill in the art. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

### Numerical Ranges

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values≧0 and ≦2 if the variable is inherently continuous. Finally, the variable can take multiple values in the range, including any sub-range of values within the cited range.

As used herein, unless specifically indicated otherwise, the word “or” is used in the inclusive sense of “and/or” and not the exclusive sense of “either/or.”

As used herein, EMR includes the range of wavelengths approximately between 200 nm and 10 mm. Optical radiation, i.e., EMR in the spectrum having wavelengths in the range between approximately 200 nm and 100 μm, is employed in some of the embodiments described above, but, also as discussed above, many other wavelengths of energy can be used alone or in combination. The actual spectrum may also include broad-band components, either providing additional treatment benefits or having no effect on treatment. Additionally, the term optical (when used in a term other than term “optical radiation”) applies to the entire EMR spectrum. Other energy may be used for treatment in similar fashion. For example, sources such as ultrasound, photo-acoustic and other sources of energy may also be used. Thus, although some embodiments described herein are described with regard to the use of EMR, other forms of energy are within the scope of the invention and the claims.

What is claimed is:

1. A method for treating tissue, comprising:
simultaneously compressing sub-volumes of the subject's skin; and
applying a thermal element proximal to the subject's skin to alter the temperature within a target depth of at least the depth of the reticular dermis, wherein the target depth is substantially adjacent the sub-volumes and the sub-volumes are separated from one another by substantially uncompressed volumes of the subject's skin.

2. The method of claim 1 wherein the target depth includes at least one of the reticular dermis, subcutaneous fat and muscle.

3. The method of claim 1 wherein the thermal element provides cooling, heating, a combination thereof, or a cycled combination thereof.

4. The method of claim 1 wherein the thermal element comprises a plurality of cooling elements adjacent a plurality of heating elements.

5. The method of claim 1 further comprising:
applying treatment radiation to at least a portion of the sub-volumes.

6. The method of claim 1 wherein one or more protrusions both compress the sub-volumes and apply the thermal element proximal to the subject's skin.

7. A device for selectively treating a subject's tissue, comprising:
a contact surface comprising two or more protrusions adapted to compress sub-volumes of a subject's skin such that the sub-volumes are separated from one another by substantially uncompressed volumes of the subject's skin; and
a thermal control element providing temperature control to at least the two or more protrusions of the contact surface, the two or more protrusions are sized to alter the temperature of the subject's tissue at a target depth of at least the depth of the reticular dermis in the region of the compressed sub-volumes.

8. The device of claim 7 wherein the two or more protrusions are sized to have a depth that range from about 0.5 mm to about 25 mm, to have a width in contact with tissue that ranges from about 0.25 mm to about 50 mm.

9. The device of claim 7 wherein the thermal control element provides cooling, heating, a combination thereof, or a cycled combination thereof.

10. The device of claim 7 wherein the thermal control element provides a plurality of protrusions adapted for heating adjacent to a plurality of protrusions adapted for cooling.

11. The device of claim 7 further comprising:
a radiation source providing treatment radiation to at least one of the two or more protrusions, the radiation source for applying treatment radiation to at least a portion of the sub-volumes.

12. The device of claim 7 wherein the contact surface is disposed on a wearable garment.

13. The device of claim 12 wherein wearing the wearable garment compresses the two or more protrusions against sub-volumes of the subject's skin.

14. The device of claim 7 wherein the contact surface is disposed on a piece of furniture.

15. The device of claim 14 wherein positioning the subject on the piece of furniture compresses the two or more protrusions against sub-volumes of the subject's skin.

16. The device of claim 7 wherein the device is a vice comprising a first side, a second side, and a mechanism for compression disposed adjacent at least one of the first and the second side, the two or more protrusions are disposed on a surface of one or more of the first side and the second side, the one or more of the first side and the second side compress the two or more protrusions against sub-volumes of the subject's skin.

17. The device of claim 16 further comprising vacuum applied to the device to increase the pressure applied to the subject's skin by the two or more protrusions.

18. The device of claim 7 wherein the device comprises a first roller, a second roller, and a mechanism for compression disposed adjacent at least one of the first roller and the second roller, the two or more protrusions are disposed on a surface of one or more of the first roller and the second roller, the rollers are adapted to roll in opposing directions to thereby compress the two or more protrusions against sub-volumes of the subject's skin.

19. The device of claim 18 further comprising vacuum applied to the device to increase the pressure applied to the subject's skin by the two or more protrusions.

20. The device of claim 7 wherein at least one of the two protrusions has a solid shape.

21. The device of claim 7 wherein at least one of the two protrusions has an at least partially hollow shape.

22. The device of claim 7 wherein at least one of the two protrusions has shape selected from the group of square, rectangular, cylindrical, spherical, and grooved.

23. The device of claim 7 wherein the device comprises a cup, two or more protrusions are disposed on the inside surface of the cup, the cup adapted to apply a vacuum to suction at least a portion of the subject's skin into the inside surface of the cup and to thereby compress the two or more protrusions against sub-volumes of the subject's skin.

24. A method for selectively treating subcutaneous tissue, comprising:
inserting a cannula to a subcutaneous region of a subject's body; and
delivering cooling to the subcutaneous tissue such that the subcutaneous tissue being treated has a temperature within the range of from about −5 C to about 20 C.

25. The method of claim 24 wherein the cannula delivers cooling to the subcutaneous tissue by thermal conduction.

26. The method of claim 24 wherein the cannula delivers a cooling agent to the subcutaneous tissue.

27. The method of claim 24 further comprising:
venting at least a portion of the cooling agent from the subcutaneous region.
